(19) United States
(12) Patent Application Publication
(10) Pub. No.: US 2024/0399172 A1
(43) Pub. Date: Dec. 5, 2024

(54) METHODS AND SYSTEMS FOR DETERMINING IRRADIATION FIELD ANGLES

(71) Applicant: SHANGHAI UNITED IMAGING HEALTHCARE CO., LTD., Shanghai (CN)

(72) Inventor: Mengrong HAN, Shanghai (CN)

(73) Assignee: SHANGHAI UNITED IMAGING HEALTHCARE CO., LTD., Shanghai (CN)

(21) Appl. No.: 18/677,869

(22) Filed: May 29, 2024

(30) Foreign Application Priority Data
May 29, 2023 (CN) 202310615883.6

(51) Int. Cl. A61N 5/10

(52) U.S. Cl. CPC A61N 5/1065; A61N 5/1031; A61N 5/1069; A61N 5/1075; A61N 5/1081

(57) ABSTRACT

Embodiments of the present disclosure provides a system and method for determining a radiation field angle. The system comprises at least one storage medium, the at least one storage medium including a set of instructions; and at least one processor in communication with the at least one storage medium, wherein when executing the set of instructions, the at least one processor is configured to cause the system to perform operations including obtaining an alternative angle set, the alternative angle set including multiple selectable beam angles; and determining a target irradiation field angle set based on the alternative angle set through iterative calculations.

### CROSS-REFERENCE TO RELATED APPLICATION

The present disclosure claims priority to Chinese application No. 202310615883.6, filed on May 29, 2023, and the contents of which are incorporated herein by reference.

### TECHNICAL FIELD

The present disclosure relates to the field of medical technology, and in particular, to a method and system for determining an irradiation field angle.

### BACKGROUND

In the radiotherapy planning process, a physician is usually required to pre-set an irradiation field angle, therefore, the choice of irradiation field angle is highly dependent on the physician's experience. However, the efficacy of the radiotherapy plan can be impacted due to the variability of cases and the movement of target areas and organs during execution. Additionally, prolonged execution of radiotherapy programs can affect the patient experience.

Therefore, it is desired to provide a method for determining an irradiation field angle considering a planned execution time, so as to select a combination of irradiation field angles with a short execution time.

### SUMMARY

One or more embodiments of the present disclosure provide a system for determining an irradiation field angle. The system includes at least one storage medium, the at least one storage medium including a set of instructions; and at least one processor in communication with the at least one storage medium. When executing the set of instructions, the at least one processor is configured to cause the system to perform operations including: obtaining an candidate alternative angle set, the candidate alternative angle set including multiple selectable beam angles; determining a target irradiation field angle set based on the candidate alternative angle set through iterative calculations, wherein each iteration of the iterative calculations includes determining a preset objective function value related to the alternative angle set, and determining the target irradiation field angle based on the preset objective function value.

In some embodiments, the selectable beam angle includes a gantry angle and/or a table angle.

In some embodiments, the selectable beam angle is generated based on a preset angle range and a preset step size.

In some embodiments, the at least one processor is further configured to determine the preset angle range and/or the preset step size based on at least one of object image data, object delineation data, or radiotherapy prescription data.

In some embodiments, the at least one processor is further configured to determine the preset angle range and/or the preset step size based on at least one of a shape of a lesion, a size of the lesion, a malignancy level of the lesion, an amount of target tissue and/or target organ, or a dange level of the lesion.

In some embodiments, the preset objective function value is determined based at least on a penalty term of an irradiation field execution time and a fluence map loss, the penalty term of the irradiation field execution time being determined based on at least one movement cost between beams corresponding to a candidate angle set of the at least one candidate angle set, and the fluence map loss being determined based on an actual dose distribution corresponding to the candidate angle set and a target dose distribution.

In some embodiments, the at least one movement cost between beams corresponding to the candidate angle set is determined by: obtaining a sorted beam angle sequence by sorting multiple selectable beam angles included in the candidate angle set based on a preset sorting rule; and determining the at least one movement cost between beams corresponding to the candidate angle set based on the sorted beam angle sequence.

In some embodiments, the determining the at least one movement cost between beams corresponding to the candidate angle set based on the sorted beam angle sequence includes: determining at least one beam angle interval value based on the sorted beam angle sequence; and determining the at least one movement cost between beams corresponding to the candidate angle set based on the at least one irradiation beam angle interval value.

In some embodiments, the determining the at least one movement cost between beams corresponding to the candidate angle set based on the sorted beam angle sequence includes: determining at least one beam-to-beam movement time based on the sorted beam angle sequence; and determining the at least one movement cost between beams corresponding to the candidate angle set based on the at least one beam-to-beam movement time.

In some embodiments, the determining the at least one movement cost between beams corresponding to the candidate angle set includes: determining the at least one movement cost between beams corresponding to the candidate angle set using an evaluation model based on the candidate angle set and the target dose distribution. The evaluation model is a machine learning model, an input of the evaluation model includes the candidate angle set and the target dose distribution, and an output of the evaluation model includes the at least one movement cost between beams corresponding to the candidate angle set.

In some embodiments, the fluence map loss is determined by: determining a fluence map loss corresponding to the candidate angle set using a fluence map loss calculation model based on the candidate angle set and irradiation parameters under each selectable beam angle in the candidate angle set, the fluence map loss calculation model being a machine learning mode. An input of the fluence map loss calculation model includes the candidate angle set and the irradiation parameters under each selectable beam angle in the candidate angle set, and an output of the fluence map loss model includes the fluence map loss corresponding to the candidate angle set.

In some embodiments, the determining the preset objective function value of the candidate angle set includes: determining the preset objective function value by a weighted sum of the penalty term of the irradiation field execution time and the fluence map loss of the candidate angle set.

In some embodiments, a weight of the fluence map loss is determined based on an excessive dose of a target tissue and a target organ.

In some embodiments, the excessive dose of the target tissue and the target organ is determined by: calculating an excessive dose at each voxel point of the target tissue and the target organ based on the actual dose distribution corresponding to the candidate angle set and the target dose distribution; and determining the excessive dose of the target tissue and the target organ by a weighted sum of the excessive dose at each voxel point. Different voxel points of the target tissue and target organ correspond to different weights.

In some embodiments, the determining the preset objective function value of the candidate angle set includes: determining the preset objective function value as a product of the penalty term of the irradiation field execution time and the fluence map loss of the candidate angle set; or determining the preset objective function value as a power of the penalty term of the irradiation field execution time of the fluence map loss of the candidate angle set.

In some embodiments, the determining the preset objective function value of the candidate angle set includes: determining the preset objective function value by applying a preset mapping to the penalty term of the irradiation field execution time and the fluence map loss of the candidate angle set.

In some embodiments, the determining the preset objective function value of the candidate angle set includes: determining the preset objective function value based on the penalty term of the irradiation field execution time, the fluence map loss of the candidate angle set, and an irradiation field number regular term.

In some embodiments, the each iteration of the iterative calculations further includes: obtaining a first candidate angle set by transforming an existing candidate angle set of a current iteration, and updating an existing candidate angle set based on the first candidate angle set; determining the preset objective function value of each candidate angle set in the existing candidate angle set; and determining a next candidate angle set to enter a next iteration based on the preset objective function value from the existing candidate angle set.

One or more embodiments of the present disclosure provide a system for determining an irradiation field angle. The system includes obtaining an alternative angle set, the alternative angle set including multiple selectable beam angles; obtaining a dose restriction condition and delineation data of a region of interest; determining a target irradiation filed angle set by optimizing the alternative angle set based on the dose restriction condition and the delineation data.

One or more embodiments of the present disclosure provide a method for determining an irradiation field angle. The method for determining a irradiation field angle comprises: obtaining an alternative angle set, the alternative angle set including multiple selectable beam angles; determining a target irradiation field angle set based on the alternative angle set through iterative calculations, each iteration of the iterative calculations includes determining a preset objective function value related to the alternative angle set, and determining the target irradiation field angle based on the preset objective function value.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an application scenario of a system for determining an irradiation field angle according to some embodiments of the present disclosure;

FIG. 2 is a schematic diagram illustrating exemplary modules of the system for determining an irradiation field angle according to some embodiments of the present disclosure;

FIG. 3 is a flowchart illustrating an exemplary process for determining an irradiation field angle according to some embodiments of the present disclosure;

FIG. 4 is a flowchart illustrating an exemplary process for determining a movement cost between beams based on a sorted beam angle sequence according to some embodiments of the present disclosure;

FIG. 5 is a schematic diagram illustrating another exemplary flowchart for determining movement cost between beams based on a sorted beam angle sequence according to some embodiments of the present disclosure;

FIG. 6 is a schematic diagram illustrating a range and step size determination model according to some embodiments of the present disclosure;

FIG. 7 is a schematic diagram of an evaluation model according to some embodiments of the present disclosure;

FIG. 8 is a schematic diagram illustrating a fluence map loss calculation model according to some embodiments of the present disclosure;

FIG. 9 is a schematic diagram illustrating a weight determination model in some embodiments of the present disclosure;

FIG. 10 is a flowchart illustrating an exemplary process for determining a target radiation field angle set based on a genetic algorithm according to some embodiments of the present disclosure;

FIG. 11 is a flowchart illustrating an exemplary process for determining a target radiation field angle set based on a pruning optimization algorithm according to some embodiments of the present disclosure; and

FIG. 12 is a flowchart illustrating an exemplary process for determining a target radiation field angle set based on a generation optimization algorithm according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with the accompanying drawings without creative labor. Unless obviously obtained from the context or the context illustrates otherwise, the same numeral in the drawings refers to the same structure or operation.

It should be understood that the terms “system”, “device”, “unit” and/or “module” as used herein is a way to distinguish between different components, elements, parts, sections or assemblies at different levels. However, the words may be replaced by other expressions if other words accomplish the same purpose.

As shown in the present disclosure and the claims, unless the context clearly suggests an exception, the words “a,” “an,” “one,” “one kind,” and/or “the” do not refer specifically to the singular, but may also include the plural. Generally, the terms “including,” and “comprising” suggest only the inclusion of clearly identified steps and elements that do not constitute an exclusive list, and the method or device may also include other steps or elements.

Flowcharts are used in the present disclosure to illustrate operations performed by a system in accordance with embodiments of the present disclosure. It should be appreciated that the preceding or following operations are not necessarily performed in an exact sequence. Instead, steps can be processed in reverse order or simultaneously. Also, it is possible to add other operations to these processes or remove a step or steps from them.

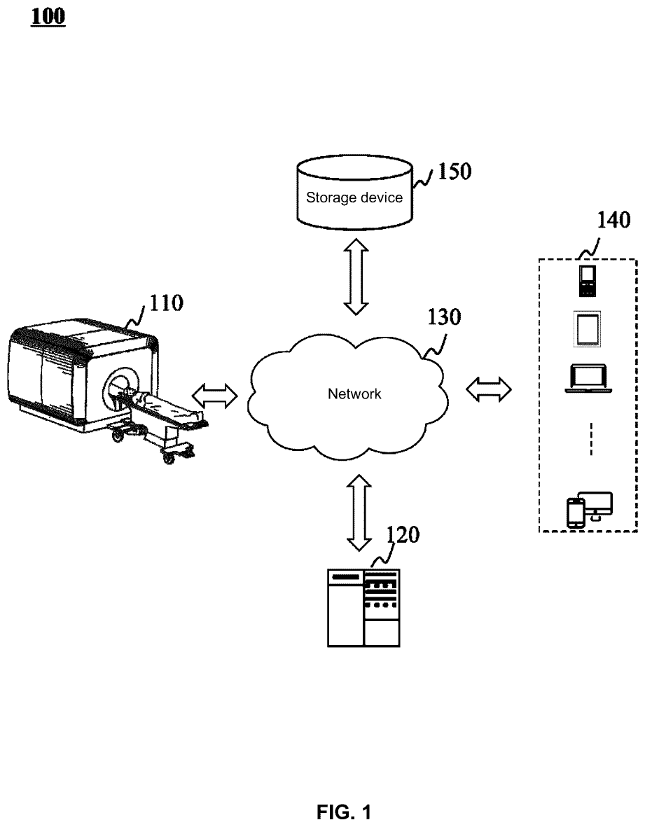

FIG. 1 is a schematic diagram illustrating an exemplary application scenario of a system for determining an irradiation field angle according to some embodiments of the present disclosure. In some embodiments, as shown in FIG. 1, components in an application scenario 100 of a system for optimizing an irradiation field angle (hereinafter referred to as the application scenario 100) may include a medical device 110, a processor 120, a network 130, a terminal device 140, and a storage medium 150. In some embodiments, components in the application scenario 100 may be connected and/or communicate with each other via the network 130 (e.g., a wireless connection, a wired connection, or a combination thereof).

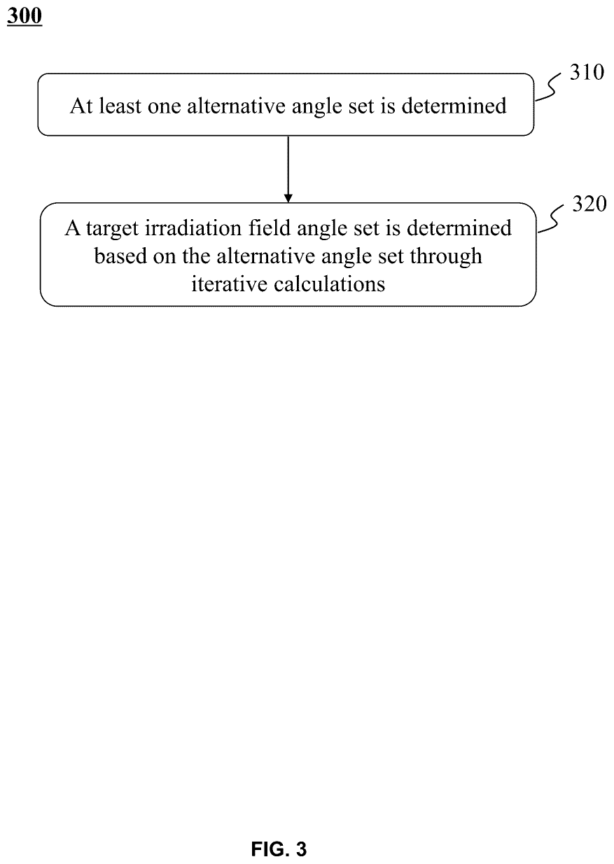

The medical device 110 refers to a device that can perform a treatment program on an object and/or capture a target image. The object may be a patient, an artificial object, an experimental object, etc. The target image may be an image containing the object. For example, the medical device 110 may administer irradiation therapy to a tumor region of the object. As another example, the medical device 110 may acquire the target image of the object, and perform the irradiation therapy on the object based on the target image. In some embodiments, the object may include a particular portion, organ, and/or tissue in a patient, an artificial object, an experimental object, etc. For example, the object may be a patient's head, chest, heart, stomach, blood vessels, soft tissues, tumors, nodules, etc., or any combination thereof. In some embodiments, the object may include a region of interest (ROI). For example, the region of interest may be a region containing the patient's lesion (e.g. a tumour, a nodule, etc.) and target tissues and/or target organs surrounding the lesion (i.e. organs and/or tissues that may be endangered by radiation therapy). More description of the lesion, the target tissues and the target organs can be found in FIG. 3 and its associated description.

In some embodiments, the medical device 110 may include one or more medical devices. In some embodiments, at least one medical device of the one or more medical devices may be used for both imaging and treatment. In some embodiments, the imaging and treatment may also be accomplished by different medical devices of the one or more medical devices.

In some embodiments, the medical device 110 may include a radiotherapy device, and the radiotherapy device may administer the irradiation therapy to the object. The radiotherapy device may include a gantry, the gantry being coupled to a treatment head. In some embodiments, the treatment head may include an irradiation source (e.g., a bulb tube and Multi Leave Collimators (MLCs). The irradiation source may emit a beam toward the object. The MLC may be used to collimate a beam emitted from the irradiation source to form a region of irradiation of a particular shape. In some embodiments, the radiotherapy device may also include a table (or couch, platform or support), a high voltage generator, a rectifier circuit, a circulating cooling device, or the like. The table is configured to support the object, which may be part of the radiotherapy equipment or may be independent of the radiotherapy equipment.

In some embodiments, the radiotherapy device may include a single-modality device or a multi-modality device. In some embodiments, the single-modality device may be configured to administer the irradiation therapy to at least a portion of the object, e.g., an x-ray therapy device, a medical electron gas pedal, etc. In some embodiments, the multi-modality device may acquire a medical image associated with at least a portion of the object and perform the irradiation therapy on at least a portion of the object. For example, a CT-guided irradiation therapy device, an MRI-guided irradiation therapy device, or the like.

In some embodiments, the medical device 110 may include an imaging device. For example, the imaging device may include one or a combination of one or more of a computed tomography imaging device (CT), a magnetic resonance imaging device (MRI), a positron emission tomography device (PET), or the like.

In some embodiments, the medical device 110 may send acquired information (e.g., the target image) to the processor 120, and the processor 120 receives information and integrates and processes the information. The medical device 110 may receive commands, etc., sent by a user (e.g., a physician) via the terminal device 140, and perform relevant operations in accordance with the commands, e.g., performing a radiotherapy program, etc. In some embodiments, the medical device 110 may be directly connected to other components in the application scenario 100. In some embodiments, the medical device 110 may exchange data and/or information with other components in the application scenario 100 (e.g., the processor 120, the terminal device 140, the storage medium 150) via the network 130. For example, the medical device 110 may transmit the acquired information to the processor 120 via the network 130. As another example, the medical device 110 may obtain commands sent by the terminal device 140 via the network 130, etc. In some embodiments, one or more components of the application scenario 100 (e.g., the processor 120, the storage medium 150) may be included within the medical device 110.

In some embodiments, the medical device 110 may receive execution instructions generated by the processor 120 and perform medical operations (e.g., scans and/or irradiation therapy, etc.) based on the execution instructions.

The processor 120 may process data and/or information related to the system for determining an irradiation field angle. For example, the processor 120 may acquire a target image captured by the medical device 110 and process the target image. As another example, the processor 120 may send a preset objective function value after processing to the storage medium 150 for saving. More description of the preset objective function value can be found in FIG. 3 and its related description. The processor 120 may also obtain pre-stored data and/or information from storage medium 150.

In some embodiments, the processor 120 may comprise one or more sub-processing devices (e.g., a single-core processing device or a multi-core processing device). By way of example only, the processor 120 may include a central processing unit (CPU), an application-specific integrated circuit (ASIC), an application-specific instruction processor (ASIP), a graphics processor (GPU), a physical processor (PPU), a digital signal processor (DSP), a field-programmable gate array (FPGA), a programmable logic circuit (PLD), a controller, a microcontroller unit, Reduced Instruction Set Computer (RISC), microprocessor, etc. or any combination of the above. The processor 120 may process data, information, and/or processing results obtained from other devices or system components and execute program instructions based on the data, information, and/or processing results to perform one or more of one or more of the functions described herein.

In some embodiments, the processor 120 may obtain instructions from the storage medium 150 to perform the following operations: determining at least one candidate angle set, each of the at least one candidate angle set including one or more selectable beam angles; and determining a target irradiation field angle set based on the at least one candidate angle set and a preset optimization algorithm. The preset optimization algorithm may comprise: determining the target irradiation field angle set through iterative calculations. Each iteration of the iterative calculations includes: determining a preset objective function value corresponding to each candidate angle set in the at least one candidate angle set, and updating the at least one candidate angle set based on the preset objective function value corresponding to the each candidate angle set to determine the target irradiation field angle set. More description of this embodiment can be found in FIG. 3 and its related description.

The network 130 may connect the components of the application scenario 100 and/or connect the system to an external resource section. The network 130 enables communication between the various components, as well as with other portions outside of the application scenario 100, to facilitate the exchange of data and/or information. In some embodiments, one or more components in the application scenario 100 (e.g., the medical device 110, the processor 120, the terminal device 140, the storage medium 150) may send data and/or information to other components over the network 130.

The terminal device 140 is a device and/or software used by the user associated with the application scenario 100. Users relevant to the application scenario 100 include but are not limited to, physicians (e.g., clinicians, radiologists), nurses, etc. For example, the terminal device 140 may be a device or software that provides control of the medical device 110, and the user may give an execute treatment plan command to the medical device 110 via the terminal device 140 to cause the medical device 110 to execute a treatment plan (e.g., a radiotherapy plan, etc.). In some embodiments, the terminal device 140 may be instructed to cause the processor 120 to perform a method for determining an irradiation field angle according to some embodiments of the present disclosure. In some embodiments, the terminal device 140 may be one of a mobile device, a tablet computer, a laptop computer, a desktop computer, and other devices with input and/or output capabilities, or any combination thereof.

The storage medium 150 may be configured to store data and/or instructions. The storage medium 150 may store data and/or information obtained from the medical device 110, the processor 120, and/or the terminal device 140. For example, the storage medium 150 may store target images obtained from the medical device 110, historical treatment data (e.g., historical target dose distribution, etc.), or the like. For example, the storage medium 150 may store data and/or information processed by the processor 120, for example, the preset objective function value, etc. In some embodiments, the storage medium 150 may store a set of instructions, and the processor 120 may perform an operation of determining the target irradiation field angle set based on the foregoing instructions. In some embodiments, the storage medium 150 may comprise mass memory, removable memory, read-write memory, read-only memory, or the like, or any combination thereof. In some embodiments, the storage medium 150 may be implemented on a cloud platform.

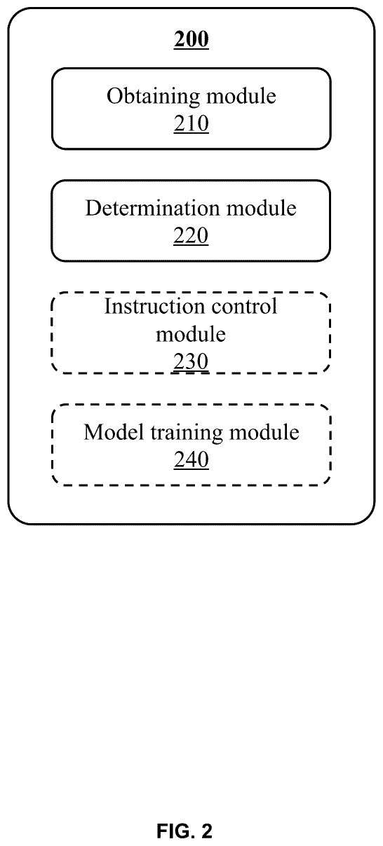

FIG. 2 is a schematic diagram illustrating exemplary modules of a system for determining an irradiation field angle according to some embodiments of the present disclosure.

In some embodiments, a system 200 for determining an irradiation field angle may include obtaining module 210 and a determination module 220, as shown in FIG. 2. In some embodiments, the system 200 for determining an irradiation field angle may be part of the processor 120.

The obtaining module 210 is configured to obtain an alternative angle set, the alternative angle set including multiple selectable beam angles.

In some embodiments, the obtaining 210 may determine at least one candidate angle set based on the alternative angle set.

In some embodiments, the obtaining 210 may select a preset number of selectable beam angles from the alternative angle set to constitute the candidate angle set.

Further description of the obtaining module 210 and the determining the at least one candidate angle set can be found in FIG. 3 and its related description.

The determination module 220 is configured to determine a target irradiation field angle set based on the alternative angle set. In some embodiments, the determination module 220 may determine the target irradiation field angle set based on at least one candidate angle set and a optimization algorithm, and the preset optimization algorithm comprises determining the target irradiation field angle set through iterative calculations. Each iteration of the iterative calculations includes: determining a preset objective function value corresponding to each candidate angle set in the at least one candidate angle set, and updating the at least one candidate angle set based on the preset objective function value corresponding to the each candidate angle set to determine the target irradiation field angle set.

In some embodiments, the determination module 220 may determine a preset objective function value based on at least a penalty term of an irradiation field execution time and a fluence map loss.

In some embodiments, the determination module 220 may obtain a sorted beam angle sequence by sorting multiple selectable beam angles included in the candidate angle set based on a preset sorting rule; and determine the at least one movement cost between beams corresponding to the candidate angle set based on the sorted beam angle sequence.

In some embodiments, the determination module 220 may utilize an evaluation model to determine the at least one movement cost between beams corresponding to the candidate angle set based on the candidate angle set and a target dose distribution.

In some embodiments, the determination module 220 may determine the preset objective function value based on the penalty term of the irradiation field execution time, a fluence map loss of the candidate angle set, and an irradiation filed number regular term.

In some embodiments, the determination module 220 may determine the preset objective function value by a weighted sum of the penalty term of the irradiation field execution time and a fluence map loss of the candidate angle set.

Further description of the determination module 220 determining the target irradiation field angle set can be found in FIG. 3 and its associated description.

In some embodiments, the system 200 for determining an irradiation field angle may further include an instruction control module 230. The instruction control module 230 is configured to generate an execution instruction related to the target irradiation field angle set and send the execution instruction to a medical device to control the medical device to perform a medical operation with the target irradiation field angle set. More description of the instruction control module 230 can be found in FIG. 3 and its related description.

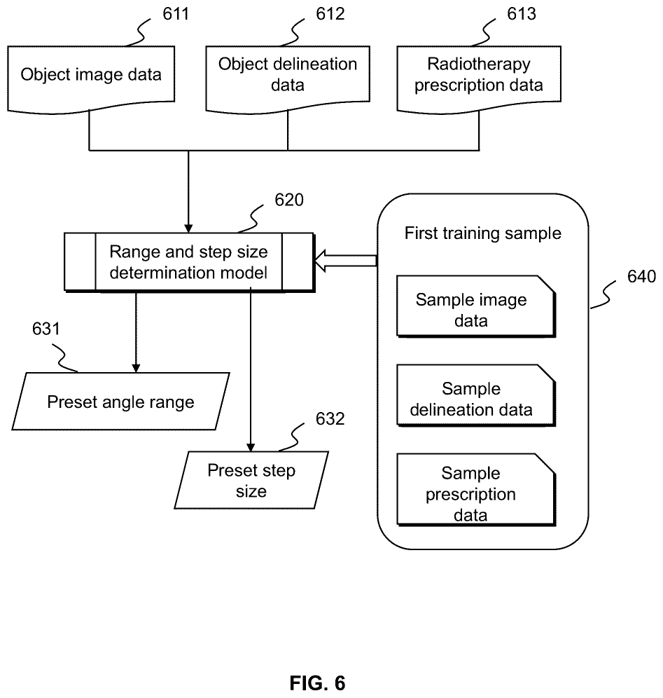

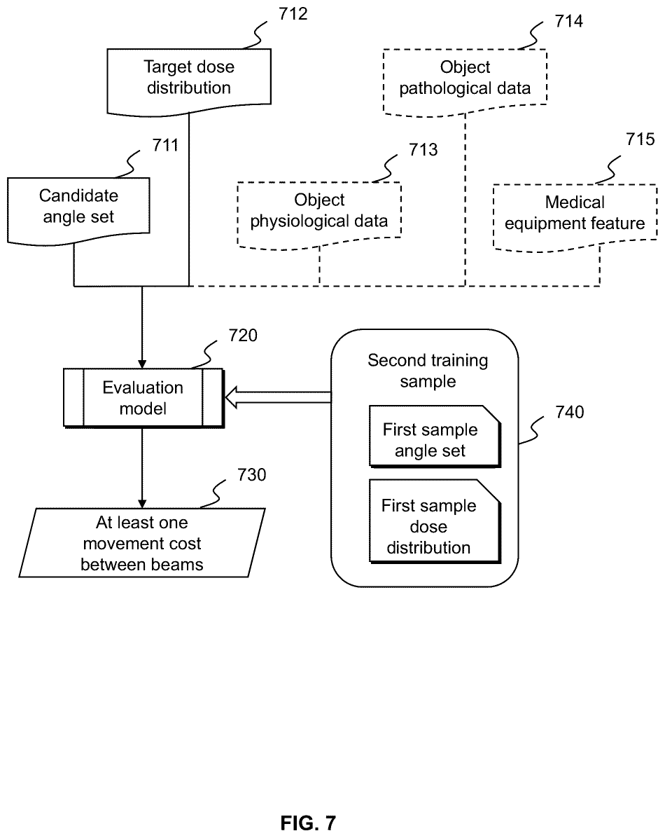

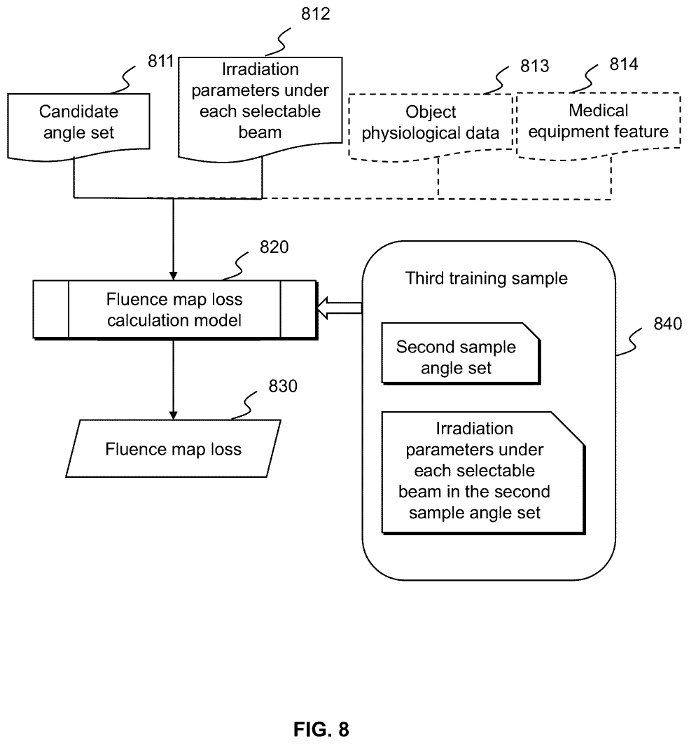

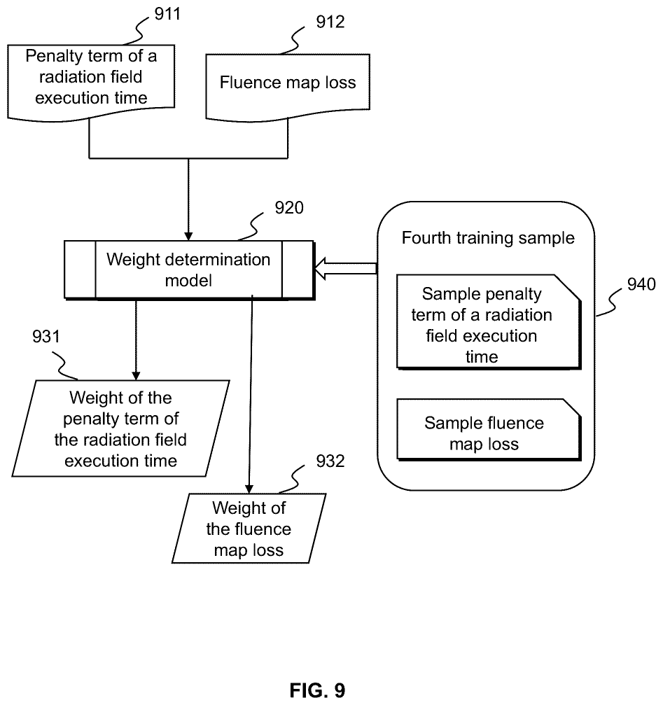

In some embodiments, the system 200 for determining an irradiation field angle may further include a model training module 240. The model training module 240 is configured to train at least one of a range and step size determination model, an evaluation model, or a fluence map loss calculation model. Further descriptions of the range and step size determination model, the evaluation model, and the fluence map loss calculation model can be found in FIG. 6, FIG. 7, FIG. 8, and/or FIG. 9 and their related descriptions.

It is to be noted that the above description of the system for determining an irradiation field angle and its modules is provided only for descriptive convenience, and does not limit the present disclosure to the scope of the embodiments cited. It is to be understood that for a person skilled in the art, with an understanding of the principle of the system, it may be possible to arbitrarily combine the modules or form a sub-system to be connected to the other modules without departing from this principle. In some embodiments, the obtaining 210, the determination module 220, and the instruction control module 230 disclosed in FIG. 2 may be different modules in a system, or they may be a single module to implement the functions of two or more of the above-described modules. For example, the individual modules may share a common storage module, and the individual modules may each have a respective storage module. Morphisms such as these are within the scope of protection of the present disclosure.

FIG. 3 is a flowchart illustrating an exemplary process for determining an irradiation field angle according to some embodiments of the present disclosure. As shown in FIG. 3, a process 300 includes following steps. In some embodiments, the process 300 may be performed by the system 200 for determining an irradiation field angle or the processor 120.

Step 310, an alternative candidate angle set is obtained. In some embodiments, step 310 may be performed by the obtaining module 210 or the processor 120.

The alternative angle set may include one or more selectable beam angles. In some embodiments, the alternative angle set may include a plurality of selectable beam angle intervals, and a boundary value of each selectable beam angle interval may be a plurality of selectable beam angles.

The selectable beam angle is a beam angle that is available when constructing a radiotherapy plan.

The radiotherapy plan is a plan for performing the irradiation therapy on the object (e.g., a patient). The radiotherapy is a very important treatment in the treatment of tumors. Prior to the radiotherapy, it is often necessary to determine a treatment plan to be used to achieve the target of treatment in order to ensure that a irradiation dose is concentrated as much as possible on a target region (e.g., a focal region) and to protect normal tissues (i.e., tissues that are not diseased) and vital organs (e.g., organs that are important for human functioning such as the heart and the liver) around the focal region so that the focal region (e.g., a tumor) is eliminated while minimizing damage to the surrounding normal tissues.

The radiotherapy plan may include one or more angles and irradiation intensities at which irradiation rays are projected, and the physician may perform the radiotherapy on the object in accordance with the radiotherapy plan. The radiotherapy plan may also include an irradiation duration of one or more irradiation rays, an irradiation interval between two adjacent irradiation rays, or the like. The radiotherapy plan may be determined based on actual treatment needs. For example, the radiotherapy plan may be determined based on object information (e.g., location of a target region, size of the target region, etc.) so that certain pre-set goals (e.g., amount of irradiation received by the target region) are met (e.g., to meet the need for treatment of the tumor area).

The beam angle is an angle at which the irradiation rays are projected. The beam angle may be a combination of angles comprising one or more beam information. The beam information may be information about an angle, e.g., the beam information may include gantry angle information, table angle information, and angle information of other relevant medical devices during execution of the radiotherapy plan, etc. In some embodiments, the selectable beam angle may include a gantry angle and/or a table angle. For example, an angle value of a beam angle α₁ may be expressed as (50°,60°), which means that a gantry angle value of the beam angle α₁ is 50° and a table angle value is 60°.

The gantry angle is a tilt/rotation angle of a gantry. The gantry may be a device (e.g., the medical device 110) or part of a device (e.g., an irradiation source in the medical device 110) that emits irradiation.

The table angle is an angle of a tilt/rotation angel of a table. In conjunction with the above, the table may be a platform on which a patient/patients, etc., are placed while undergoing the radiotherapy.

In some embodiments, the selectable beam angle may be generated based on a preset angle range and a preset step size.

The preset angle range is a preset angle range of beam angles that may be used to perform the radiotherapy plan. In some embodiments, the preset angle range may include a preset gantry angle range and/or a preset table angle range. For example, the preset gantry angle range and the preset table angle range may be any angle range interval, for example, [0°,45°], [0°,90°], [0°,360°], [10°, 60°], etc. The preset angle range (i.e., the preset gantry angle range and the preset table angle range) may be determined automatically by a user or by the system.

The preset step size is an interval between preset angle values. In some embodiments, the preset step may include a press gantry angle step and/or a preset table angle step. The preset step size may be automatically determined by the user or the system. For example, the preset step size may be 100 (i.e., the preset gantry angle step size and the preset table angle step size are both 10°).

For example, assuming that the preset gantry angle range is [00,3600], the preset table angle range is [0°,90°], the preset gantry angle step size is 10°, and the preset table angle step size is 10°, then the gantry angle values generated include 0°, 10°, 20°, . . . , 350°, 360°, and the table angle values generated include 0°, 10°, 20°, . . . , 80°, 90°, based on which each of the gantry angle values may be sequentially paired with each of the table angle values, and thus (0°,0°), (0°,10°), . . . , (0°,90°), . . . , (360°,0°), (360°,10°), . . . , (360°,90°), a total of 37×10 angles may be generated, which the 37×10 angles constitute all selectable beam angles.

As another example, assuming the preset gantry angle range be [0°,360°], the preset table angle range be [0°,90°], the preset gantry angle step size be 10°, and the preset table angle step size be 10°, then gantry angle values that may be generated include 0°, 10°, 20°, . . . , 350°, 360°, and table angle values that may be generated include 0°, 10°, 20°, . . . , 80°, 90°, on the basis of which an obtained gantry angle interval may be [0°,10°], [10°,20°], [20°,30°], . . . , [350°,360°], and an obtained table angle interval may be [0°, 10°], [10°, 20°], [20°,30°], . . . , [80°,90°], and each of the gantry angle interval may be paired in turn with each of the table angle interval in turn, which in turn may generate ([0°,10°],[0°,10°]), ([0°,10°],[10°,20°]), . . . , ([0°,10°],[80°,90°]), ([10°,20°],[0°,10°]), ([10°,20°],[10°,20°]), . . . , ([10°,20°],[80°,90°]), . . . , ([[350°,360°],[0°,10°]), ([350°,360°],[10°,20°]), . . . , ([350°,360°],[80°,90°]), for a total of 36×9 selectable beam angle intervals, which constitutes all selectable beam angle intervals.

The alternative angle set may also be generated in other ways, for example, by randomly generating a preset number of non-repeating angles within a preset angle range to constitute the alternative angle set, which is not limited here.

In some embodiments, the obtaining module 210 or the processing device (e.g., the processor 120) may determine the preset angle range and/or the preset step size based on at least one of object image data, object delineation data, or radiotherapy prescription data.

In some embodiments, the obtaining module 210 or the processing device (e.g., the processor 120) may determine the preset angle range and/or the preset step size based on at least one of the object image data, the object delineation data, or the radiotherapy prescription data using a range and step size determination model.

The object image data refers to a scanned image including a lesion of an object. For example, the scanned image, may be a CT image including the lesion of the object and a target tissue and/or a target organ. For another example, an image of a region of interest. The lesion refers to a target site within the object that requires a radiotherapy. For example, the lesion may be a cancerous lesion. The target tissue refers to a tissue surrounding the lesion. A distance between the target tissue and the lesion is less than a first distance threshold. The target tissue is yet not a target lesion since the target tissue is still a normal tissue surrounding the lesion. The target organ refers to a vital organ surrounding the lesion and/or is not yet a target lesion since the target organ is still a normal organ surrounding the lesion. A distance between the lesion and the target organ is less than a second distance threshold. The target organ has a high impact on body function (e.g., heart, etc.). The first distance threshold and/or the second distance threshold may be preset. The target lesion refers to a lesion that requires a radiotherapy. For example, the target lesion is a cancerous lesion. In some embodiments, the first distance threshold and the second distance threshold may be the same or different. For example, the first distance threshold and the second distance threshold may both be preset to be 15 cm, or one of the first distance threshold and the second distance threshold may be 10 cm and the other may be 12 cm. For example, in the case where the lesion is located in the right lobe of the liver, and both the first distance threshold and the second distance threshold are preset to be 20 cm, the target tissue may include the left lobe of the liver, the hepatic vein, the hepatic artery, and the duodenum, and the target organ may include the gallbladder.

The object delineation data refers to contour data of the lesion and the target tissue and/or the target organ in the object image data. The object delineation data may be obtained by a developer or implementer (e.g., a physician) of a radiotherapy plan outlining a contour of the lesion and the target tissue and/or the target organ based on the object image data, and the outlining may be performed based on circling or stroking or marking, etc. For example, if the object image data includes a lesion located in the right lobe of the liver, and the first distance threshold and the second distance threshold are both preset to be 20 cm, then the target tissue may include the left lobe of the liver, the hepatic vein, the hepatic artery, and the duodenum, and the target organ may include the gallbladder, based on which the physician may outline a contour of the lesion, the left lobe of the liver, the hepatic vein, the hepatic artery, the duodenum, and the gallbladder through stroking, thereby obtaining corresponding object delineation data.

The radiotherapy prescription data includes a preset irradiation dose and a first dose ceiling. The preset irradiation dose refers to an irradiation dose to be applied to the lesion during a current radiotherapy procedure in the radiotherapy plan, and the first dose ceiling refers to an upper limit of an irradiation dose of the target tissue and the target organ (an irradiation dose received by the target tissue and the target organ during a radiotherapy procedure may not exceed the upper limit). Dose is an amount of irradiation energy deposited per unit mass of target material. For example, the radiotherapy prescription data may be (2Gy, 0.2Gy), which represents that a preset irradiation dose is 2Gy and a surrounding dose ceiling is 0.2Gy.

In some embodiments, the radiotherapy prescription data may also include a dose restriction condition of a region of interest. The region of interest refers to a region containing the lesion and its surrounding target tissues and/or target organs. The dose restriction condition includes a lesion dose lower limit (an irradiation does received by the lesion is no less than the lower limit) and a second dose upper limit, and the lesion dose lower limit refers to a lower value below which the irradiation dose received by the lesion cannot fall, and the second dose upper limit refers to an upper value above which the irradiation dose received by a delineated target tissue and/or target organ cannot rise. For example, the dose restriction condition may be ‘the lesion lower dose limit is 2 Gy and the second dose upper limit is 0.1 Gy’.

The range and step size determination model is a machine learning model. An input to the range and step size determination model may include the object image data, the object delineation data, and the radiotherapy prescription data, and an output of the range and step size determination model may include a preset angle range and a preset step size. More description of the range and step size determination model can be found in FIG. 6 and its related description.

In some embodiments, the obtaining module 210 or the processing device (e.g., processor 120) may determine the preset angle range and/or the preset step size based on at least one of a shape of a lesion (represented based on a cross-sectional roundness value of the lesion), a size of the lesion (represented by a volume of the lesion), a malignancy of the lesion (represented by a malignancy level grade, e,g, grade 1, grade 2 and grade 3 in increasing order of malignancy), a number of target tissues and/or target organs, and a risk level of the lesion. A shape of the lesion and a size of the lesion may be determined based on the object image data (e.g., a CT image) and the object delineation data.

In some embodiments, a span of the preset angle range may be positively correlated with the shape of the lesion, i.e., the larger the cross-sectional roundness value of the lesion, the larger the span of the preset angle range. For example, when the cross-sectional roundness value of the lesion is in a range of 0 to 0.2 cm, the span of the preset angle range is 60°; when the cross-sectional roundness value of the lesion is in a range of 0.2 cm to 0.4 cm, the span of the preset angle range is 120°; and when the cross-sectional roundness value of the lesion is in a range of 0.4 to 0.6 cm, the span of the preset angle range is 180°, etc.

The span of the preset angle range includes a span of the preset gantry angle range and/or a span of the preset table angle range. The span of the preset gantry angle range refers to a difference between a right boundary value and a left boundary value of the preset gantry angle range. Similarly, the span of the preset table angle range refers to a difference between a right boundary value and a left boundary value of the preset table angle range.

In some embodiments, the span of the preset angle range may be positively correlated with the size of the lesion, i.e., the larger the volume of the lesion, the larger the span of the preset angle range. For example, when the volume of the lesion is less than 20 cm³, the span of the preset angle range is 60°; when the volume of the lesion is in a range of 20 cm³ to 40 cm³, the span of the preset angle range is 120°; and when the volume of the lesion is in a range of 40 cm³ to 60 cm³, the span of the preset angle range is 180°, etc.

In some embodiments, the span of the preset angle range may be positively correlated with the malignancy of the lesion, i.e., the higher the malignancy of the lesion, the larger the span of the preset angle range. For example, when the malignancy of the lesion is grade 1, the span of the preset angle range is 90°; when the malignancy of the lesion is grade 2, the span of the preset angle range is 180°; and when the malignancy of the lesion is grade 3, the span of the preset angle range is 360°.

In some embodiments, the span of the preset angle range may be positively correlated with the number of target tissues and/or target organs. For example, in the case where the lesion is located in the right lobe of the liver, and both the first distance threshold and the second distance threshold are preset to be 20 cm, then the target tissue may include the left lobe of the liver, the hepatic vein, the hepatic artery, and the duodenum (a number of the target tissues is 4), and the target organ may include the gallbladder (a number of the target organ is 1), based on which, the number (or count) of target tissues and/or target organs is 5. Exemplarily, when the number of normal tissues around the lesion is 1, the span of the preset angle range is 60°; when the number of normal tissues around the lesion is 2, the span of the preset angle range is 120°; and when the number of normal tissues around the lesion is 3, the span of the preset angle range is 180°, etc.

In some embodiments, the span of the preset angle range may be positively correlated with the risk level of the lesion. The risk level of the lesion may include low danger, intermediate danger, and high danger (corresponding to the period of onset in which the lesion is located as early, intermediate, and late, respectively, with increasing risk levels). For example, when the risk degree of the lesion is a low danger, the span of the preset angel range is 60°; when the risk level of the lesion is a medium danger, the span of the preset angle range is 120°; when the risk level of the lesion is a high danger, the span of the preset angle range is 180°.

In some embodiments, the preset step size may be negatively correlated with the shape of the lesion, i.e., the larger the cross-sectional roundness value of the lesion, the smaller the preset step size. For example, when the cross-sectional roundness value of the lesion is in a range of 0 to 0.2 cm, the preset step size is 30°; when the cross-sectional roundness value of the lesion is in a range of 0.2 cm to 0.4 cm, the preset step size is 25°; and when the cross-sectional roundness value of the lesion is in a range of 0.4 to 0.6 cm, the preset step size is 20°, etc.

In some embodiments, the preset step size may be negatively correlated with the size of the lesion, i.e., the larger the volume of the lesion, the smaller the preset step size. For example, when the volume of the lesion is less than 20 cm³, the preset step size is 30°; when the volume of the lesion is in a range of 20 cm³ to 40 cm³, the preset step size is 25°; when the volume of the lesion is in a range of 40 cm³ to 60 cm³, the preset step size is 20°, etc.

In some embodiments, the preset step size may be negatively correlated with the malignancy of the lesion, i.e., the higher the malignancy of the lesion, the smaller the preset step size. For example, when the malignancy of the lesion is grade 1, the preset step size is 20°; when the malignancy of the lesion is grade 2, the preset step size is 10°; and when the malignancy of the lesion is grade 3, the preset step size is 5°.

In some embodiments, the preset step size may be negatively correlated with the number of target tissues and/or target organs. For example, when a number of normal tissues surrounding the lesion is 1, the preset step size is 30°; when a number of normal tissues surrounding the lesion is 2, the preset step size is 25°; when a number of normal tissues surrounding the lesion is 3, the preset step size is 20°, etc.

In some embodiments, the preset step size may be negatively correlated with the risk level of the lesion. For example, the preset step size is 30° when the risk level of the lesion is a low danger, the preset step size is 20° when the risk level of the lesion is a medium danger, and the preset step size is 10° when the risk level of the lesion is a high danger.

In some embodiments of the present disclosure, by the above-described manner for determining the preset angle range and/or the preset step size, the adaptability of a determined preset angle range and/or preset step size can be improved, such that the determined preset angle range and/or preset step size can be better adapted to meet the needs of an irradiation treatment performed on a patient.

Step 320, the target irradiation field angle set is determined based on the alternative angle set through iterative calculations. In some embodiments, the step 320 may be executed by the determination module 220.

In some embodiments, the obtaining module 210 or the processing device (e.g., the processor 120) may determine at least one candidate angle set based on the alternative angle set

The candidate angle set refers to a set consisting of one or more selectable beam angles included in a particular radiotherapy plan. Different candidate angle sets may each correspond to a particular radiotherapy plan. The candidate angle set may be a subset of an alternative angle set. For example, if the alternative angle set is {α₁,α₂,α₃,α₄,α₅,α₆}, the candidate angle set may be {α₁,α₃}, {α₂,α₃,α₅,α₆}, {as}, and so on, where α₁, α₂, α₃, α₄, α₅, α₆ are selectable beam angles.

In some embodiments, the processing device (e.g., the processor 120) may determine at least one candidate angle set from the alternative angle set based on a preset generation condition.

The preset generation condition may be set based on rules. In some embodiments, the preset generation condition may be that a preset number of selectable beam angles from the alternative angle set are selected to form a candidate angle set. For example, a preset number of beam angles are selected from the candidate angle set at an equal interval, starting with a certain beam angle. Exemplarily, a process for determining the at least one candidate angle set based on the preset generation condition is as follows: if the alternative angle set comprises (0°, 0°), (0°, 10°), . . . , (0°, 90°), (10°, 0°), (10°, 10°), (10°, 20°), . . . , (10°, 90°), (20°, 0°), . . . , (350°, 80°), (350°, 90°), (360°, 0°), (360°, 10°), . . . , (360°, 90°), a total of 37×10 angles, and the preset generation condition is to select one beam angle from the alternative angle set in a preset order (e.g., an order of beam angles in the alternative angle set in the above example) at intervals of one beam angle, and each of the five selected beam angles constitutes a candidate angle set: {(0°, 0°), (0°, 20°), (0°, 40°), (0°, 60°), (0°, 80°)}; then, according to the rule, starting from different angles, one beam angle is selected for every interval of 1 beam angle, and every 5 selected beam angles constitute a candidate angle set, then a number of different candidate angle sets may be generated.

The preset generation condition may also be set based on information about the object. For example, based on the relevant information of the object (e.g., a location, size, and malignancy of a tumor in the patient's body, etc.), a number of appropriate angles are first screened (e.g., based on a rule constructed based on the physician's experience, based on a result of angles selected for the same or similar objects in historical data, and randomly selected among remaining angles after removing one or more angles based on the relevant information of the object, etc.) from the alternative angle set, and then, from the angles, a preset number of beam angles are determined (e.g., randomly selected, sequentially selected from the smallest angle/maximum angle after being sorted according to the size of the angles, selected based on angles selected for the same or similar objects in the historical data, etc.), then the candidate angle set is constituted. Another example is to randomly select a preset number of non-repeating selectable beam angles from the alternative angle set, and determine whether the preset number of non-repeating selectable beam angles satisfies a radiotherapy condition (e.g., the preset number of non-repeating selectable beam angles may completely cover a tumor region of the patient), and, in response to a determination that the radiotherapy condition is satisfied, determine the preset number of non-repeating selectable beam angles to be the candidate angle set; and in response to a determination that the radiotherapy condition fails to be satisfied, do not determining the preset number of non-repeating selectable beam angles as the candidate angle set, and re-selecting a preset number of non-repeating selectable beam angles after judgment until a preset number of non-repeating selectable beam angles selected satisfies the radiotherapy condition.

The candidate angle set may also be generated in other ways, for example, by randomly selecting a preset number of non-repeating selectable beam angles in the alternative angle set to directly constitute the candidate angle set, which is not limited herein.

In some embodiments, the determination module 210 or the processing device (e.g., the processor 120) may determine a preset number based on vector matching.

In some embodiments, the determination module 210 or the processing device (e.g., the processor 120) may determine the preset number based on through vector matching based on a preset irradiation dose and a target dose distribution.

The dose distribution is a distribution of doses received by at least one voxel site in a target (e.g., a lesion). The dose distribution may be represented as vectors, matrices (where each element represents doses received at a voxel point), or in other ways.

The voxel point is used to represent a smallest unit in a three-dimensional space (similar to a pixel point in a two-dimensional space). The voxel point refers to a point on a target (e.g., a lesion or an object) after the target (e.g., a lesion or an object) has been abstracted into a 3D model (e.g., by modeling software).

The target dose distribution refers to an irradiation dose to be received at each voxel point contained in a preplanned target (e.g., a lesion). It is to be understood that a sum of irradiation doses to be received at all voxel points in the target dose distribution is equal to the preset irradiation dose.

The target dose distribution may be data in the form of vectors. For example, the target dose distribution may be (51.1, 52, 50.9, . . . , 53, 52.5), characterizing an irradiation dose to be received at each voxel point included in a preplanned target (e.g., a lesion) as 51.1cGy, 52cGy, 50.9cGy, . . . , 53cGy, and 52.5cGy.

In some embodiments, the determination module 210 or the processing device (e.g., the processor 120) may determine a reference vector based on a historical preset irradiation dose and a historical target dose distribution of a historical object and construct a vector database based on the reference vector. Further, the processing device may determine a target vector based on a currently preset irradiation dose and a target dose distribution of a target object (i.e., an object currently in need of radiotherapy); based on the target vector, determine at least one reference vector (i.e., vector matching) in the vector database that satisfies a preset vector condition; determine the preset angle range and/or the preset step size based on the at least one reference vector. The historical object refers to an object of a historical irradiation treatment process, e.g., a cancer patient who has undergone irradiation treatment, etc. The historical object may include objects that are the same as or different from the target object.

The reference vector is a vector obtained by combining the historical preset irradiation dose and the historical target dose distribution of the historical object. For example, if a historical preset irradiation dose and a historical target dose distribution for a particular radiotherapy or scan in the history of a historical object are 2Gy and (51.1, 52, . . . 53, 52.5), respectively, the reference vector obtained by merging may be (2, 51.1, 52, . . . , 53, 52.5).

The vector database means a database that includes reference vectors for multiple historical objects.

The target vector refers to a vector obtained by combining t the currently preset irradiation dose and the target dose distribution of the target object. For example, if the currently preset irradiation dose and the target dose distribution of the target object are 2Gy and (53.1, 50.9, . . . , 53, 51.5), the target vector obtained by merging may be (2, 53.1, 50.9, . . . , 53, 51.5).

The preset vector condition may be that a vector distance between the target vector and the reference vector is less than a third distance threshold. The vector distance may be a Euclidean distance of the vector, a cosine distance, etc. The third distance threshold may be manually preset.

In some embodiments, each reference vector may have a corresponding set of reference data. The reference data may include a reference number and the reference number may be set artificially.

In some embodiments, the determination module 210 or the processing device (e.g., the processor 120), after determining the at least one reference vector based on the vector matching, may calculate a mean value of a preset number of reference data corresponding to the at least one reference vector and round (e.g., upwardly) the mean value as the preset number.

In some embodiments, the determination module 210 or the processing device (e.g., the processor 120) may determine the preset number based on a clustering analysis.

The clustering analysis refers to a process of clustering objects based on a clustering algorithm and determining the preset number based on a clustering result. Types of clustering algorithms may include a variety of types, for example, K-Means (K-means) clustering, density-based clustering methods (DBSCAN), or the like. A clustering object of the clustering analysis may include a target vector and a plurality of clustering vectors. More description of the target vectors can be found in the previous description. Clustering metrics of the clustering analysis may include the preset irradiation dose and the target dose distribution.

A clustering vector means a vector constructed based on the merging of the historical preset irradiation dose, the historical target dose distribution, and a historical preset number of the historical object. For example, a hisyorical preset irradiation dose, a historical target dose distribution, and a historical preset number corresponding to a certain irradiation treatment or scan of the historical object are 2Gy, (53.1, 50.9, . . . , 53, 51.5) and 3, then the clustering vector obtained by merging may be (2, 53.1, 50.9, . . . , 53, 51.5, 3).

In some embodiments, after completing the clustering to obtain at least one cluster, the determination module 210 or the processing device (e.g., the processor 120) may determine a cluster containing the target vector as a target cluster and determine the preset number based on a historical preset number of cluster vectors contained in the target cluster. For example, the processing device (e.g., the processor 120) may determine the plural or mean value(rounded) of a historical preset number contained in the cluster vector in the target cluster as the preset number.

In some embodiments, the determination module 210 or the processing device (e.g., the processor 120) may determine the preset number based on an inhomogeneity of the target dose distribution.

The inhomogeneity of the target dose distribution may be characterized based on a standard deviation of an irradiation dose at each voxel point in the target dose distribution.

In some embodiments, the preset number may be positively correlated with the inhomogeneity of the target dose distribution, i.e., the larger the value of the standard deviation of the irradiation dose at each voxel point in the target dose distribution, the larger the preset number.

In some embodiments, after determining the preset number based on the manner described above, an operator of the medical device 110 (e.g., a physician performing a radiotherapy) may manually adjust the preset number, e.g., by increasing the preset number by 1 or the like.

In some embodiments of the present disclosure, the generation of the candidate angle set facilitates the determination of beam angle combinations for radiotherapy plans that are superior in terms of both dose and cost of execution, and facilitates safeguarding the effectiveness of irradiation therapy while enhancing the experience of a treatment subject.

In some embodiments of the present disclosure, by the manner for determining the candidate angle set and a number thereof, the adaptability of a determined candidate angle set can be enhanced, which can help to subsequently determine a target irradiation field angle set from the determined candidate angle set.

The target irradiation field angle set refers to an optimal angle set determined from the alternative angle set (e.g. an angle set made of multiple selectable beam angles that minimise damage to normal tissue around the lesion, maximise coverage of the target area, etc.).

In some embodiments, the processing device 120 may determine the target irradiation field angle set based on a preset optimization algorithm.

The preset optimization algorithm refers to an algorithm that is pre-set in an irradiation field angle optimization system for determining the target irradiation field angle set. In some embodiments, the processing device (e.g., the processor 120) may access steps and/or instructions corresponding to the preset optimization algorithm via a storage device (e.g., the storage medium 150) or otherwise (e.g., via the terminal device 140), and determine the target irradiation field angle set by executing the steps and/or instructions.

In some embodiments, the preset optimization algorithm may be preset based on historical experience. In some embodiments, the preset optimization algorithm may comprise a calculation. The calculation comprising: determining a preset objective function value corresponding to each candidate angle set in the at least one candidate angle set; determining the target irradiation field angle based on the preset objective function value corresponding to each candidate angle set.

A preset objective function refers to a function that is used to determine a degree of superiority or inferiority of the candidate angle set.

The preset objective function may be constructed based on some preset objectives during a radiotherapy process. For example, if the preset objective includes that an irradiation received in the target region (e.g., a tumor or a region surrounding the tumor, or another region of interest in the patient) reaches a preset irradiation dose value, and a treatment time of the radiotherapy is less than a preset time value, the preset objective function may be constructed based on the irradiation received in the target region, and the treatment time of the radiotherapy. For example, the preset objective function is constructed as: ƒ=Mx+Ny, where, x denotes the irradiation received in the target region, y denotes the treatment time of the radiotherapy, and M and N denote corresponding coefficients, which may be obtained by preset or determined by simulation and modeling methods, actual experimental methods and so on.

The preset objective function value refers to a value of the preset objective function. The processing device (e.g., the processor 120) may input at least the candidate angle set into the preset objective function to obtain the preset objective function value. The preset objective function value may be used to characterize the degree of superiority of the candidate angle set, e.g., the smaller the preset objective function value of the candidate angle set, the better the candidate angle set (e.g., when the radiotherapy is administered to the object based on beam angles included in that candidate angle set, the object receives less irradiation dose from the radiotherapy or less damage to normal tissues of the object, etc.).

In some embodiments, the preset objective function value may be set based on an application scenario of irradiation field angle optimization and relevant information about the object. For example, based on the application scenario of the irradiation field angle optimization (e.g., the need to irradiate as much of the patient's tumor as possible), relevant information about the object (e.g., the location and size of the tumor in the patient's body), and the irradiation coverage of the patient's tumor region based on the candidate angle set, the preset objective function value is determined (e.g., if the inverse of the irradiation coverage is determined to be the preset objective function value, then the smaller the preset objective function value, the better preset objective function value).

In some embodiments, the preset objective function value may be determined at least based on a penalty term of an irradiation field execution time and a fluence map loss. For example, the preset objective function value may be a sum of the penalty term of the irradiation field execution time and the fluence map loss.

In some embodiments, the penalty term of the irradiation field execution time is determined based on at least one movement cost between beams corresponding to the candidate angle set.

The movement cost between beams is a cost of moving the beam (equivalent to the irradiation ray) from one beam angle to another beam angle. The cost may be expressed in a variety of ways, such as a time cost, an energy cost, etc. For example, a movement cost between beams of a beam from a beam angle α₁ to a beam angle α₂ may be a movement time of the beam from the beam angle α₁ to the beam angle α₂. As another example, a movement cost between beams of the beam from the beam angle α₁ to the beam angle α₂ may be an energy cost (e.g., power cost) of moving the beam from the beam angle α₁ to the beam angle α₂.

In some embodiments, the penalty term of the irradiation field execution time may be a sum of each of the at least one movement cost between beams corresponding to the candidate angle set. For example, the penalty term of the irradiation field execution time may be expressed by a following Equation (1):

$${F}_{b e a m} = \sum {C}_{i} \qquad ( 1 )$$

Where, F_{beam} denotes a penalty term of an irradiation field execution time corresponding to a certain candidate angle set, and Cᵢ denotes an i-th movement cost between beams (i is a positive integer) out of at least one movement cost between beams corresponding to the candidate angle set.

In some embodiments, the penalty term of the irradiation field execution time may be a weighted sum of each of the at least one movement cost between beams corresponding to the candidate angle set. For example, the penalty term of the irradiation field execution time may be expressed by a following Equation (2):

$${F}_{b e a m} = \sum {k}_{i} {C}_{i} \qquad ( 2 )$$

Where, F_{beam} denotes a penalty term of an irradiation field execution time corresponding to a certain candidate angle set, Cᵢ denotes the i-th movement cost between beams out of at least one movement cost between beams corresponding to the candidate angle set, and kᵢ denotes a weighting factor corresponding to the i-th movement cost between beams (i is a positive integer).

The penalty term of irradiation field execution time may also be the sum, a weighted sum, a product, a weighted product, or any other mathematical operation characterizing a positive correlation, of one or more of the at least one movement cost between beams corresponding to the candidate angle set (e.g., F_{beam}−Σe^{c}^{ᵢ}, F_{beam} denotes the penalty term of irradiation field execution time corresponding to the candidate angle set, Ci denotes the i-th movement cost between beams out of at least one movement cost between beams (i takes a positive integer), and e is a natural constant), which is not limited herein.

In some embodiments, the processing device (e.g., the processor 120) may determine a first execution beam angle and a last execution beam angle from the candidate angle set; and determine a movement cost between beams for moving from the first execution beam angle to the last execution beam angle as the at least one movement cost between beams corresponding to the candidate angle set. The first execution beam angle and the last execution beam angle are determined according to the actual radiotherapy plan. By determining the movement cost between beams for moving from the first execution beam angle to the last execution beam angle as the at least one movement cost between beams, it is possible to reduce the complexity of the quantification of the at least one movement cost between beams while maintaining a sufficient degree of accuracy of the quantification; furthermore, constructing the penalty term of the irradiation field execution time based on the at least one movement cost between beams helps to more efficiently determine the optimal combination of beam angles with a short execution time.

For example, a candidate angle set A includes a selectable beam angle α₁, a selectable beam angle α₂, a selectable beam angle α₃, and a selectable beam angle α₄, i.e., A={α₁, α₂, α₃, α₄}, and if the first execution beam angle is α₃, the last execution beam angle is α₂, then at least one movement cost between beams corresponding to the candidate angle set A may include: a movement cost between beams of a beam for moving from the selectable beam angle α₃ to the selectable beam angle α₂. It is noted that a number of selectable beam angles that may be included in the candidate angle set is not limited to four, but may be an arbitrary number.

In some embodiments, the processing device (e.g., the processor 120) may obtain a sorted beam angle sequence by sorting multiple selectable beam angles included in the candidate angle set based on a preset sorting rule; and determine the at least one movement cost between beams corresponding to the candidate angle set based on the sorted beam angle sequence.

The sorted beam angle sequence is an ordered sequence formed by arranging one or more beam angles in some order.

In some embodiments, the preset sorting rule may be to sort based on one or more of a magnitude of a beam angle value, a sequence of beam angles being performed in a radiotherapy plan, and an adjacency between beam angles. Exemplarily, taking the preset sorting rule as sorting based on the sequence of beam angles being performed in the radiotherapy plan as an example, assuming a candidate angle set A={(10°, 90°), (50°, 40°), (20°, 70°), (50°, 80°)}, and if, according to the radiotherapy plan, the order of execution of each beam angle in the radiotherapy plan is: (10°, 90°), (20°, 70°), (50°, 40°), (50°, 80°), then the candidate angle set is sorted based on the preset sorting rule, and a sorted beam angle sequence of beam angles is ((10°, 90°), (20°, 70°), (50°, 40°), (50°, 80°)).

In some embodiments, the processing device (e.g., the processor 120) may determine a movement cost between beams between each two adjacent beam angles in the sorted beam angle sequence as the at least one movement cost between beams corresponding to the candidate angle set.

For example, if the candidate angle set A includes the selectable beam angle α₁, the selectable beam angle α₂, the selectable beam angle α₃, and the selectable beam angle α₄, i.e., A={α₁, α₂, α₃, α₄}, and a sorted beam angle sequence corresponding to the candidate angle set A is (α₂, α₃, α₁, α₄), then the at least one movement cost between beams corresponding to the candidate angle set A includes: a movement cost between beams of a beam for moving from the selectable beam angle α₂ to the selectable beam angle α₃, a movement cost between beams of a beam for moving from the selectable beam angle α₃ to the selectable beam angle α₁, and a movement cost between beams of a beam for moving from the selectable beam angle α₁ to the selectable beam angle α₄.

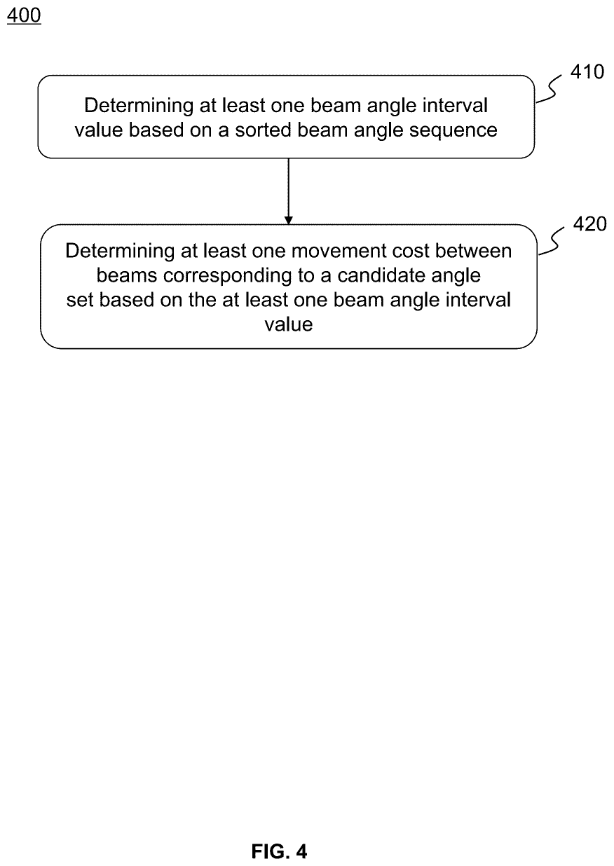

In some embodiments, the processing device (e.g., the processor 120) may determine at least one beam angle interval value based on the sorted beam angle sequence; and determine the at least one movement cost between beams corresponding to the candidate angle set based on the at least one beam angle interval value. Further description of the beam angle interval value, a manner for determining the beam angle interval value, and determining the movement cost between beams based on the beam angle interval value can be found in FIG. 4 and its associated description.

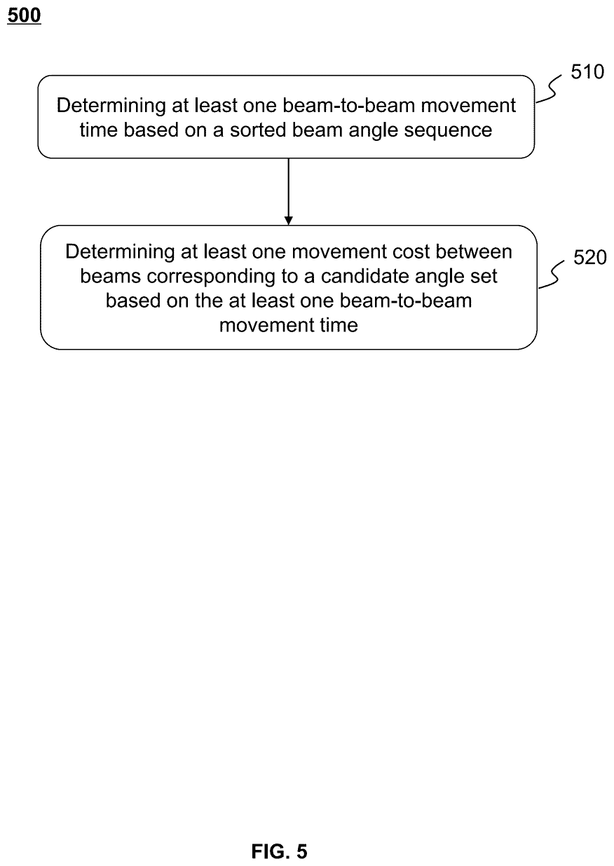

In some embodiments, the processing device (e.g., the processor 120) may determine at least one beam-to-beam movement time based on the sorted beam angle sequence; and determine the at least one movement cost between beams corresponding to the candidate angle set based on the at least one beam-to-beam movement time. The beam-to-beam movement time is determined based on trajectory planning information. The trajectory planning information includes velocity planning information for the beam-to-beam movement and acceleration planning information for the beam-to-beam movement. Further description of the beam-to-beam movement time, the trajectory planning information, determining the movement cost between beams based on the beam-to-beam movement time, or the like may be found in FIG. 5 and its related description.

In some embodiments of the present disclosure, the at least one movement cost between beams is determined based on the sorted beam angle sequence obtained after sorting based on the preset sorting rule, such that the movement cost between beams may be determined based on an optimized beam angle sequence, thereby reducing an unnecessary movement cost due to a beam angle execution sequence, facilitating a more accurate response to an execution time situation, and facilitating the generation of a more optimal combination of beam angles with a short execution time of the radiotherapy plan.

In some embodiments, the processing device (e.g., the processor 120) may utilize an evaluation model to determine the at least one movement cost between beams corresponding to the candidate angle set based on the candidate angle set and the target dose distribution. Further instructions for determining the at least one movement cost between beams corresponding to the candidate angle set using the evaluation model can be found in FIG. 7 and its associated description.

The fluence map loss is a difference or error in the radiotherapy metric. The radiotherapy metric may be flux (or a flux distribution) and/or dose (or a dose distribution).

The flux refers to a number of radioactive particles passing through a unit area per unit time, and aflux distribution refers to a distribution of flux received by at least one voxel point in a target (e.g., an object, a target region, and/or normal tissue surrounding a lesion). The flux distribution may be represented as vectors, matrices (where each element represents the flux received by a voxel point), or otherwise.

The flux and dose may be obtained by converting them to each other through a flux-dose conversion function (which can be obtained by empirical formulas, calculations based on physical laws, or based on simulations).

In some embodiments, the fluence map loss may be determined based on an actual dose distribution corresponding to the candidate angle set and the target dose distribution.

Exemplarily, the fluence map loss may be obtained by the following Equation (3):

$${F}_{d} = Ω ( d ( Φ ) , {d}_{0} ) \qquad ( 3 )$$

Where, ψ denotes the actual flux distribution, d denotes the flux-dose conversion function, d(Φ) denotes the actual dose distribution, d₀ denotes the target dose distribution, and Ω denotes a loss calculation function. The target dose distribution may be set according to the actual needs. For example, the target dose distribution may be set according to a maximum dose constraint limit. The loss calculation function may be set according to the actual optimization needs, for example, the loss calculation function may be: Ω(d(Φ), d₀)=max{d(Φ)−d₀, 0}, or Ω(d(Φ), d₀)=(max{d(Φ)−d₀, 0})².

In some embodiments, the loss calculation function may calculate the fluence map loss in any number of ways, e.g., as an absolute value of the difference, as an absolute value of the squared difference, or the like.

The actual dose distribution is a distribution of dose received by the target (e.g., the target region) during the execution of the radiotherapy plan. In some embodiments, the actual dose distribution may include dose received at the at least one voxel point.

In conjunction with the above, the flux and the dose may be converted to each other by the flux-dose conversion function, based on which the actual flux distribution and the actual dose distribution may also be converted to each other by the flux-dose conversion function.

The actual flux distribution may be obtained by Flux Map Optimization (FMO) or a Direct Aperture Optimization (DAO) algorithm based on the candidate angle set. For example, based on the one or more selectable beam angles included in the candidate angle set, a preferred irradiation parameter (irradiation duration, irradiation intensity, etc.) of a radiotherapy device at each of the selectable beam angles is obtained by the flux map optimization, and based on the irradiation parameter at each of the selectable beam angles included in the candidate angle set, the actual flux distribution is calculated.

Exemplarily, according to one or more selectable beam angles included in the candidate angle set, the preferred irradiation parameter of the radiotherapy device at each selectable beam angle obtained by the flux map optimization may be: inputting the candidate angle set and a flux optimization objective into a flux map optimization model, and the flux map optimization model outputting the preferred irradiation parameter corresponding to each selectable beam angle in the candidate angle set. The flux optimization objective may include an irradiation flux value required to be received by various portions of the target region (e.g., a tumor within the patient). The flux optimization objective may be obtained by converting through the flux-dose conversion function based on the target dose distribution. The flux map optimization model may include machine learning models, empirical formulas, computational formulas constructed based on physical laws, models obtained based on simulation, optimization algorithms (e.g., genetic algorithms), or the like.

In some embodiments, based on the candidate angle set and the irradiation parameter at each selectable beam angle in the candidate angle set, a flux distribution calculation model is utilized to determine an actual flux distribution corresponding to the candidate angle set; and based on the actual flux distribution, the actual dose distribution corresponding to the candidate angle set is determined. For more information about the fluence map loss calculation model, please see FIG. 8 and its description.

In some embodiments, the determination module 220 or the processing device (e.g., the processor 120) may determine the preset objective function value by a weighted sum of the penalty term of the irradiation field execution time and the fluence map loss of the candidate angle set.

In some embodiments, the preset objective function value may be determined by the following Equation(4):

$$f = α {F}_{b e a m} + β {F}_{d} \qquad ( 4 )$$

Where, ƒ denotes the preset objective function value, F_{beam} denotes the penalty term of the irradiation field execution time, and F_{d} denotes the fluence map loss; and α and β are weights of the penalty term of the irradiation field execution time and the fluence map loss, respectively. Values of α and β may be set artificially or calculated according to the actual demand. For example, the values of α and β may be calculated based on a difference between a previous preset objective function value result and a desired result by an optimization algorithm or calculated based on a preset rule. As another example, the values of a may be greater than β when focusing more on optimizing the penalty term of the irradiation field execution time, and the values of α and β may be the same when optimizing both the penalty term of the irradiation field execution time and the fluence map loss.

In some embodiments, the weight of the loss flux is determined based on an excessive dose of a target tissue and a target organ. In conjunction with the above, the target tissue is a normal tissue surrounding the lesion (tissues without the target lesion) and the target organ is a normal organ surrounding the lesion (organs without the target lesion) and/or vital organs.

The excessive dose refers to an irradiation dose received by the target tissue or target organ over a standard dose (referred to as being excessive). The standard dose may be preset by a radiotherapy planner (e.g., physician) based on experience.

In some embodiments, the weight of the fluence map loss may be positively correlated with the excessive dose.

In some embodiments, the determination module 220 or the processing device (e.g., the processor 120) may calculate an excessive dose for each voxel point in the target tissue and/or target organ based on the actual dose distribution and the target dose distribution corresponding to the candidate angle set. Further, the determination module 220 or the processing device may determine the excess dose of the target tissue and/or the target organ by a weighted sum of the excessive dose at each voxel point. Weights corresponding to voxel points in different target tissues and/or target organs are different.

An excessive dose at a voxel point (hereafter referred to as a voxel excessive dose) refers to an excessive irradiation dose received at a voxel point in the target tissue or the target organ. The voxel excessive dose may be calculated as follows: if an actual dose received by a voxel is greater than a target dose, then the voxel excessive dose is equal to a difference obtained by subtracting the target dose from the actual dose; if the actual dose received by the voxel is less than or equal to the target dose, then the voxel excess dose is 0.

When calculating the excessive dose, a weight corresponding to the voxel excessive dose of each voxel point is determined based on the target tissue and/or target organ to which the each voxel point belongs. For example, if the lesion is in the left lobe of the liver, the target tissue may include the right lobe of the liver, the hepatic artery, the hepatic vein, and the duodenum, and the target organ may include the gallbladder, then in calculating the excessive dose, a weight corresponding to the voxel excessive dose of voxel points within the left lobe of the liver is different from a weight corresponding to the voxel excessive dose of the voxel point within the duodenum. Further, weights corresponding to voxel excessive dose of voxel points of different target tissues or target organs may be determined based on a functional state of the target tissues or target organs. For example, the worse the functional state of the target tissue or the target organ, the greater the weight corresponding to the voxel excessive dose of voxel points within the target tissue or the target organ. Exemplarily, following the above example, if an object suffers from a liver disease and the gallbladder of the object is not diseased, then a weight corresponding to an excessive voxel dose of voxel points of the right liver is greater than a weight corresponding to an excessive voxel dose of voxel points of the gallbladder.

In some embodiments, the weight of the penalty term of the irradiation field execution time and the weight of the fluence map loss may be determined by a simulation modelling approach, an actual experimental approach, or the like.

In some embodiments, the weight of the penalty term of the irradiation field execution time and the weight of the fluence map loss may be determined based on a weight determination model. The weight determination model is a machine learning model, an input to the weight determination model may include the penalty term of the irradiation field execution time and the fluence map loss, and an output may include the weight of the penalty term of the irradiation field execution time and the weight of the fluence map loss. More description of the weight determination model can be found in FIG. 9 and its associated description.

In some embodiments, an initial preset objective function value may be determined based on one of the above two covariates (i.e., the penalty term of the irradiation field execution time and the fluence map loss), and the determined initial preset objective function value may be later adjusted based on the other covariate to obtain a final preset objective function value. For example, the preset objective function value may be determined by the following Equation (5) and Equation (6):

$${f}_{1} = {g}_{1} ( {F}_{1} ) \qquad ( 5 )$$

$$f = {g}_{2} ( {f}_{1} , {F}_{2} ) \qquad ( 6 )$$

where ƒ denotes a preset objective function value, F₁ denotes one of the penalty term of the irradiation field execution time and the fluence map loss, and F₂ denotes the other of the penalty term of the irradiation field execution time and the fluence map loss (i.e., if F₁ is the penalty term of the irradiation field execution time, then F₂ is the fluence map loss; and vice versa if F₂ is the penalty term of the irradiation field execution time, then F₁ is the fluence map loss); and g₁ and g₂ are the mapping functions, which can be preset, the For example, g₁ may be a calculated square, g₂ may be a weighted summation of the covariates, etc.

In some embodiments of the present disclosure, by the above manner for setting the weight of the fluence map loss and the weight of the penalty term of the irradiation field execution time, an evaluation focus of the candidate angle set may be adjusted according to the actual situation of each target object (e.g., the patient), on the basis of which a target irradiation field angle set that is suitable for the target object may be determined.

In some embodiments, the determination module 220 or the processing device (e.g., the processor 120) may determine the preset objective function value as a product of the penalty term of the irradiation field execution time and the fluence map loss of the candidate angle set. For example, the preset objective function value may be determined by the following Equation (7):

$$f = {F}_{b e a m} \times {F}_{d} \qquad ( 7 )$$

Where, ƒ denotes the preset objective function value, F_{beam} denotes the penalty term of the irradiation field execution time, and F_{d} denotes the fluence map loss.

In some embodiments, the determination module 220 or the processing device (e.g., the processor 120) may determine the preset objective function value as a power of the penalty term of the irradiation field execution time of the flux loss of the candidate angle set. For example, the preset objective function value may be determined by the following Equation (8):

$$f = {F}_{d}^{{F}_{b e a m}} \qquad ( 8 )$$

Where, ƒ denotes the preset objective function value, F_{beam} denotes the penalty term of the irradiation field execution time, and F_{d} denotes the fluence map loss.

In some embodiments, the determination module 220 or the processing device (e.g., the processor 120) may determine the preset objective function value by applying a preset mapping to the penalty term of the irradiation field execution time and the fluence map loss of the candidate angle set. For example, the preset objective function value may be determined by the following Equation (9):

$$f = g ( {F}_{b eam} , {F}_{d} ) \qquad ( 9 )$$

Where, ƒ denotes the preset objective function value, F_{beam} denotes the penalty term of the irradiation field execution time, F_{d} denotes the fluence map loss, and g denotes a mapping function corresponding to the preset mapping, which is monotonic in both the F_{beam} direction and F_{d} direction.

In some embodiments of the present disclosure, by the above manner for determining the preset objective function value, the preset objective function value obtained can be made reasonable and in line with the practical needs.

In some embodiments, the determination module 220 or the processing device (e.g., the processor 120) may determine the preset objective function value based on the penalty term of the irradiation field execution time, the fluence map loss of the candidate angle set, and an irradiation filed number regular term. For example, the preset objective function value may be a sum of the penalty term of the irradiation field execution time, the flux loss, and the irradiation filed number regular term. In some embodiments, the preset objective function value may be determined by the following Equation(10):

$$f = α {F}_{b e a m} + β {F}_{d} + γ {F}_{p} \qquad ( 10 )$$

Where, ƒ denotes the preset objective function value, F_{beam} denotes the penalty term of the irradiation field execution time, F_{d} denotes the fluence map loss, and Fₚ denotes the irradiation field number regular term; α, β, and γ are weights of the penalty term of the irradiation field execution time, the fluence map loss, and the irradiation field number regular term, respectively. Values of α, β, and γ may be set artificially or calculated according to the actual demand. For example, the value of a may be greater than β and γ when focusing more on optimizing the penalty term of the irradiation field execution time; or the values of α, β, and γ may be the same when optimizing the penalty term of the irradiation field execution time, the fluence map loss of the candidate angle set, and the irradiation filed number regular term in a balanced manner. As another example, based on the difference between the previous preset objective function value result and the desired result to be calculated by the optimization algorithm or calculated based on a preset rule.

In some embodiments, the irradiation field number regular term may be constructed based on an irradiation field number. The irradiation field number refers to a number of beam angles in the candidate angle set.

In some embodiments, the irradiation field number regular term may be constructed based on the irradiation field number through a preset transformation rule. For example, it is straightforward to designate the irradiation field number as the irradiation field number regular term (i.e., the preset transformation rule is the equivalence transformation), and to designate m (m takes a positive real number) power of the irradiation field number as the irradiation field number regular term (i.e., the preset transformation rule is the fetch power transformation), and so on.

In some embodiments, the irradiation field number regular term may be constructed based on the actual flux distribution. In some embodiments, the irradiation field number regular term may be constructed by the following Equation (11):

$${F}_{p} = {‖ Φ ‖}_{p} \qquad ( 11 )$$

Where, Fₚ denotes the irradiation field number regular term, Φ denotes the actual flux distribution, and ∥Φ∥ₚ denotes the p-parameter of the actual flux distribution Φ (p may take any positive real number).

In some embodiments, the p-parameter of the actual flux distribution Φ may be expressed by the following Equation (12):

$${‖ Φ ‖}_{p} = {( {\sum }_{i = 1}^{N} {\vert {v}_{i} \vert}^{p} )}^{1 / p} \qquad ( 12 )$$

Where, ∥Φ|ₚ denotes the p-paradigm of the actual flux distribution Φ, vᵢ (i=1, . . . ,N) denotes an element of the actual flux distribution (e.g., a vector element or a matrix element), and p denotes an arbitrary positive real number (e.g., p=1 for 1-paradigm, p=2 for 2-paradigm, etc.).

In some embodiments of the present disclosure, optimization of the number of beam angles can be achieved by incorporating the irradiation field number regular term into the preset objective function, so as to optimize a combination of beam angles with a suitable number of beams, which results in a high efficiency of the optimization, and a scheme that is scalability of the program.

In some embodiments, the preset optimization algorithm comprises: determining the target irradiation field angle set through iterative calculations. Each iteration of the iterative calculations includes: determining a preset objective function value corresponding to each candidate angle set in the at least one candidate angle set, and updating the at least one candidate angle set based on the preset objective function value corresponding to the each candidate angle set to determine the target irradiation field angle set.

The updating includes deleting an existing candidate angle set and/or identifying a new angle set as the candidate angle set. Exemplarily, the existing candidate angle set that is deleted may be a candidate angle set made of multiple selectable beam angles for which the preset objective function value is greater than a first function value threshold.

In some embodiments, when the iterative calculation is complete, the determination module 220 or the processing device (e.g., the processor 120) may determine a candidate angle set with a smallest preset objective function value to be the target irradiation field angle set. The end of the iterative calculation may be signified by a number of iterations of the iterative calculation reaching a preset threshold number of iterations, or the preset objective function values of all candidate angle sets obtained by the iterative calculation being less than the first function value threshold, etc.

In some embodiments, at each iteration in the iterative calculation, the determination module 220 or the processing device (e.g., the processor 120) may obtain a first candidate angle set by transforming an existing candidate angle set of a current iteration, and updated an existing candidate angle set based on the first candidate angle set. Further, the determination module 220 or the processing device may determine the preset objective function value of each candidate angle set in the existing candidate angle set; and determine a next candidate angle set to enter a next iteration based on the preset objective function value from the existing candidate angle set.

In some embodiments, the transformation comprises at least one of adjusting at least one selectable beam angle in the candidate angle set, and exchanging a selectable beam angle at the same location in two different candidate angle sets.

The adjustment may include adjusting a selected selectable beam angle to a preset angle value, or enlarging or contracting the selected selectable beam angle. For example, the adjustment may be to adjust the selected selectable beam angle to (60°,60°). As another example, the adjustment may be an increase of the preset angle value (e.g., an increase of 5°) for the selected selectable beam angle. Again, for example, the adjustment may be a reduction of the selected selectable beam angle by a preset factor (e.g., to ½). The manner of selecting the selectable beam angle from the candidate angle set may be random or may be by a preset selection manner, and an exemplary preset selection manner may be as follows: selecting a last selectable beam angle in the candidate angle set.

An exemplary process of exchanging the selectable beam angle at the same location in two different candidate angle sets may be as follows: assuming that there exists a candidate angle set A={α₁,α₂,α₃,α₄,α₅,α₆} and a candidate angle set B={α₇,α₈,α₉,α₁₀,α₁₁,α₁₂}, then α₁ and α₇ are selectable beam angles at the same location (both located at a first position in their respective candidate angle set), similarly, α₂ and α₈ are selectable beam angles at the same location, and α₃ and α₉ are selectable beam angles at the same location. . . . Based on this, for example, by exchanging α3 with α₉, the first candidate angle set obtained is A1={α₁,α₂,α₉,α₄,α₅,α₆} and B1={α₇,α₈,α₃,α₁₀,α₁₁,α₁₂}.

In some embodiments, a manner for determining the next candidate angle set to enter the next iteration from the existing candidate angle set may be as follows: N existing candidate angles with a smallest preset objective function value are determined to be a candidate angle set for entering the next iteration of calculation, where N is the number of candidate angle sets at the beginning of the current iteration of calculation.

In some embodiments of the present disclosure, by means of the iterative calculation manner described above, a set comprising selectable beam angles with a smallest preset objective function value may be determined from the alternative angle set as much as possible and subsequently determined as the target irradiation field angle, such that the adaptability of a determined target irradiation field angle set to the current patient is sufficiently improved.

In some embodiments, the preset optimization algorithm may include a heuristic manner. Heuristic algorithms are, for example, genetic algorithms, particle swarm algorithms, ant colony algorithms, or the like.

In some embodiments, the preset optimization algorithm may include a greedy strategy algorithm. The greedy strategy algorithm is, for example, generative manners, pruning optimization algorithms, domain search manners, or the like.

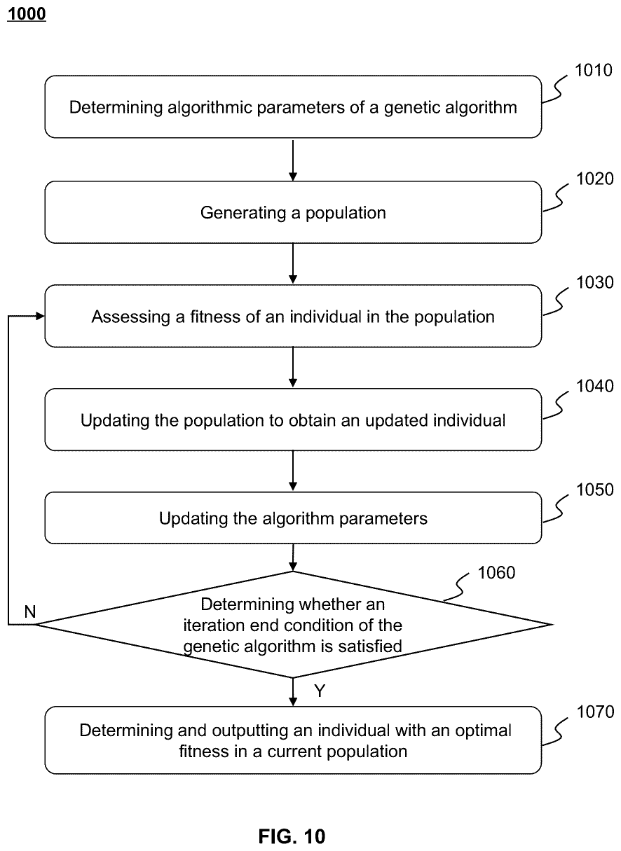

In some embodiments, the preset optimization algorithm may include a genetic algorithm. See FIG. 10 and its related descriptions for more on the genetic algorithm and determining the target irradiation field angle set based on the genetic algorithm.

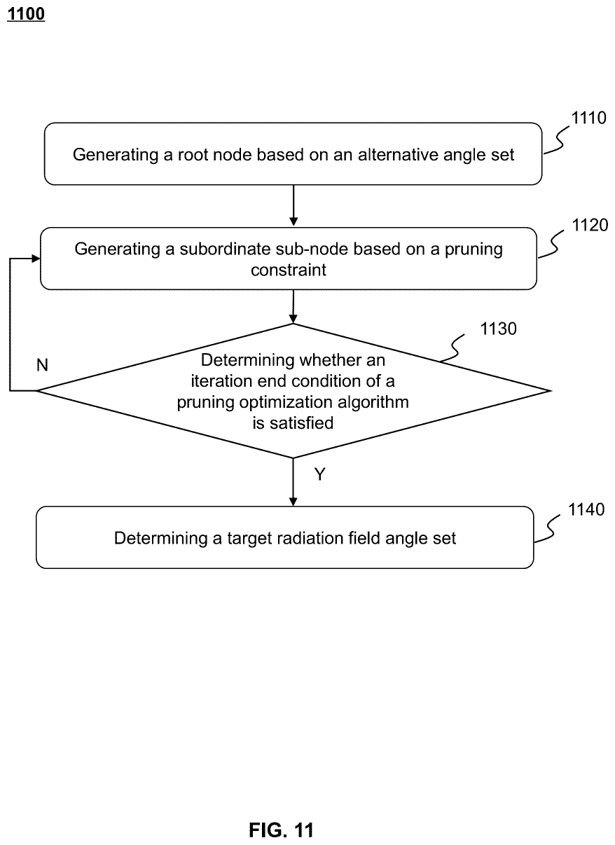

In some embodiments, the preset optimization algorithm may include a pruning optimization algorithm. For more on the pruning optimization algorithm and determining the set of target field angles based on the pruning optimization algorithm, see FIG. 11 and its related description.

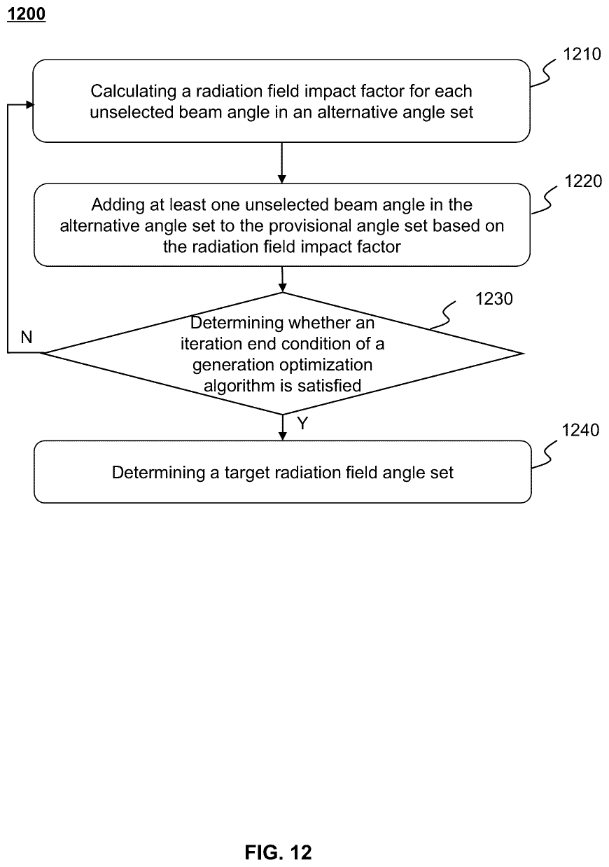

In some embodiments, the preset optimization algorithm may include a generative manner. The generative manner (also known as constructive manner, building manner) is a search optimization algorithm that adds elements one by one according to the degree of influence of each element on an objective value, thus obtaining a preferred result. Steps of the generative manner mainly include calculating the influence degree of the currently selectable element, selecting the element based on the influence degree, and judging the end condition of the iteration. For more on determining the target irradiation field angle set based on the generative manner, please see FIG. 12 and its related description.

In some embodiments, after determining the target irradiation field angle set, the instruction control module 230 may generate an execution instruction related to the target irradiation field angle set and send the execution instruction to a medical device to control the medical device to perform a medical operation (e.g., scanning and/or radiotherapy) at the target irradiation field angle set.

The execution instruction related to the target irradiation field angle set refers to a computer instruction that contains the target irradiation field angle set. The medical device (e.g., the medical device 110) may perform scanning and/or radiotherapy on the target object based on the target irradiation field angle set after receiving the execution instruction associated with the target irradiation field angle set.

In some embodiments, the determination module 220 or the processor 120 may directly optimize the alternative angle set to determine the target irradiation field angle set. For example, determining the target irradiation field angle set in a manner that traverses at least a portion of subsets of the alternative angle set, each subset includes one or more selectable beam angles. Specifically, determining all subsets of the alternative angle set; selecting (e.g., manually, randomly, etc.) some or all of the subsets; calculating a preset objective function values corresponding to each selected subset, respectively; and determining an optimal subset (a subset with a smallest function value) as the target irradiation field angle set. The preset objective function value of the subset may be calculated in the same way as that of the candidate angle set, which can be referred to the relevant description in the previous section.

In some embodiments, the determination module 220 or the processor 120 may obtain the dose restriction condition and the delineation data of the region of interest; optimize the alternative angle set based on the dose restriction condition and the delineation data to determine the target irradiation field angle set. For example, following the previous example, a subset whose corresponding actual dose distribution not satisfy the dose restriction condition is eliminated, and preset objective function values are determined for remaining subsets, and an optimal (with a smallest preset objective function value) subset is then determined to be the target irradiation field angle set. For example, after calculating a preset objective function values corresponding to each selected subset, sorting subsets involved in the calculation based on the order of the preset objective function values from the smallest to the largest, verifying whether an actual dose distribution corresponding to each of the subsets satisfy the dose restriction condition according to a sorting result, and determining a subset that satisfies the dose restriction condition with a smallest preset objective function value as the target irradiation field angle set. More descriptions of the dose restriction condition and the delineation data can be found in the previous section.

In some embodiments of the present disclosure, based on the above-described manner for determining an irradiation field angle, by optimizing a selectable beam angle set to reflect the dose of the radiotherapy plan, and using the movement cost between beams to reflect an execution cost (time, energy, etc.) of the radiotherapy plan, a combination of beam angles for the radiotherapy plan that is better in terms of both the dose and the execution cost can be generated, which can safeguard the effectiveness of the radiotherapy plan and at the same time optimize the patient's treatment experience.

It should be noted that the foregoing description of the process 300 is intended to be exemplary and illustrative only and does not limit the scope of application of the present disclosure. For a person skilled in the art, various corrections and changes can be made to the process 300 under the guidance of the present disclosure. However, these corrections and changes remain within the scope of the present disclosure. For example, the preset objective function value is equal to the sum of the penalty term of the irradiation field execution time and the fluence map loss of the candidate angle set.

FIG. 4 is a flowchart illustrating an exemplary process for determining a movement cost between beams based on a sorted beam angle sequence according to some embodiments of the present disclosure. In some embodiments, process 400 may be executed by a processing device (e.g., processor 120). As shown in FIG. 4, process 400 includes the following operations.

In 410, at least one beam angle interval value is determined based on a sorted beam angle sequence.

The beam angle interval value is used to describe a distance between two beam angles. In some embodiments, the beam angle interval value may be represented by an angular difference.

In some embodiments, the determining the at least one beam angle interval value based on the sorted beam angle sequence may include determining an angular difference between every two adjacent beam angles in the sorted beam angle sequence; and determining one or more angular differences as the at least one beam angle interval value.

Merely by way of example, the sorted beam angle sequence is set to be (α₂, α₃, α₁, α₄), then the at least one beam angle interval value includes an angular difference between a beam angle α₂ and a beam angle α₃, an angular difference between the beam angle α₃ and a beam angle α₁, and an angular difference between the beam angle α₁ and a beam angle α₄, that is, three beam angle interval values are obtained.

In some embodiments, an angle value in response to the beam angle includes a gantry angle value and a table angle value, and the angular difference may be determined based on a gantry angular difference, and a table angular difference. For example, the angular difference between the beam angle α₁ and the beam angle α₂ may be a greater value of the gantry angular difference between the beam angle α₁ and the beam angle α₂ and the table angular difference between the beam angle α₁ and the beam angle α₂.

In some embodiments, an angle value in response to the beam angle includes a gantry angle value and a table angle value, and the angular difference may be determined based on the gantry angular difference, the table angular difference, a gantry angle penalty value, and a table angle penalty value. Merely by way of example, the angular difference may be determined based on the gantry angular difference, the table angular difference, the gantry angle penalty value, and the table angle penalty value, by the following Equation (13):

$$H = max ( {ω}_{1} \vert {G}_{i} - {G}_{j} \vert , {ω}_{2} \vert {T}_{i} - {T}_{j} \vert ) \qquad ( 13 )$$

H denotes an angular difference between a beam angle i and a beam angle j, Gᵢ denotes a gantry angular value of the beam angle i, Gⱼ denotes a gantry angle value of the beam angle j, Tᵢ denotes a table angle value of the beam angle i, Tⱼ denotes a table angle value of the beam angle j, ω₁ denotes the gantry angle penalty value, ω₂ denotes the table angle penalty value, and max denotes a maximum value function. Values of ω₁ and ω₂ may be set artificially or automatically according to the actual demand. For example, when focusing on optimizing the gantry angular difference, the value of ω₁ may be greater than the value of ω₂. As another example, the values of ω₁ and ω₂ may be the same when optimizing both in a balanced manner.

In 420, at least one movement cost between beams corresponding to a candidate angle set is determined based on the at least one beam angle interval value.

In some embodiments, the processing device may determine each of the at least one beam angle interval value as each of the at least one movement cost between beams. For example, three beam angle interval values are determined as movement costs between beams corresponding to the candidate angle set. That is to say, the candidate angle set corresponds to the three movement costs between beams.

Further, the processing device may obtain a penalty term of a radiation field execution time based on the at least one movement cost between beams corresponding to the candidate angle set. Exemplarily, the radiation field execution time penalty term may be expressed by the following Equation (14):

$${F}_{b e a m} = {\sum }_{( i , j ) ∈ R} {H}_{ij} \qquad ( 14 )$$

Hᵢⱼ denotes a beam angle interval value between a beam angle i and a beam angle j, and R denotes an adjacent angle set.

The adjacent angle set refers to a set consisting of ordered pairs of every two adjacent beam angles in the sorted beam angle sequence corresponding to the candidate angle set. For example, a candidate angle set A is {α₁, α₂, α₃, α₄}, and a sorted beam angle sequence B corresponding to the candidate angle set A is (α₂, α₃, α₁, α₄), and the adjacent angle set is {(α₂, α₃), (α₃, α₁), (α₁, α₄)}.

In some embodiments of the present disclosure, based on the sorted beam angle sequence, at least one beam angle interval value is determined. The at least one beam angle interval value is further used to determine the at least one movement cost between beams corresponding to the candidate angle set, thereby allowing the angular difference to reflect an execution spend of a radiation treatment program, and quantifying the movement cost between beams efficiently. Further, the penalty term of the radiation field execution time is determined based on the movement cost between beams, which helps to efficiently determine an optimal combination of radiation field angles with a short execution time.

FIG. 5 is a schematic diagram illustrating another exemplary flowchart for determining movement cost between beams based on a sorted beam angle sequence according to some embodiments of the present disclosure. In some embodiments, process 500 may be executed by a processing device (e.g., the processor 120). As shown in FIG. 5, process 500 includes the following operations.

In 510, at least one beam-to-beam movement time is determined based on a sorted beam angle sequence. The beam-to-beam movement time is determined based on trajectory planning information. The trajectory planning information includes velocity planning information of beam-to-beam movement and acceleration planning information of beam-to-beam movement.

The beam-to-beam movement time refers to a movement time of a beam (e.g., a radiation ray) moving from one beam angle to another.

The trajectory planning information is used to describe planning information related to a process of beam movement. The trajectory planning information may include the velocity planning information of beam-to-beam movement and the acceleration planning information of beam-to-beam movement.

The velocity planning information of beam-to-beam movement may include a preset smooth motion velocity, etc. The preset smooth motion velocity refers to a preset velocity at which the beams move at a uniform velocity after completing acceleration.

The acceleration planning information of beam-to-beam movement may include an acceleration velocity at preset acceleration, an acceleration velocity at preset deceleration, etc. The preset acceleration velocity at acceleration refers to a preset acceleration velocity at the beginning of beam movement. The preset acceleration velocity at deceleration refers to a preset acceleration velocity after the beam completes uniform movement and starts to decelerate.

In some embodiments, an angle value in response to the beam angle includes a gantry angle value and a table angle value, and the beam-to-beam movement time may be determined based on a gantry movement time and a table movement time. For example, the beam-to-beam movement time from the beam angle α₁ to the beam angle α₂ may be the greater one of a gantry movement time from the beam angle α1 to the beam angle α₂ and a table movement time from the beam angle α₁ to the beam angle α₂.

The gantry movement time refers to a movement time for a gantry to move from a gantry angle value at one beam angle to a gantry angle value at another beam angle. In some embodiments, the gantry movement time may be determined based on the trajectory planning information and a preset movement rule. The preset movement rule is used to describe rules to be followed for the movement of the gantry and/or the table. For example, the preset movement rule may be used to accelerate the gantry and/or the table to the preset smooth movement velocity, or as much velocity as possible below the preset smooth movement velocity, at the preset acceleration velocity at acceleration for a certain period of time at a uniform velocity (which may be 0, i.e., a direct deceleration rather than a uniform velocity). Then, the velocity is reduced to 0 at a preset acceleration velocity at deceleration, and the beam-to-beam movement is just completed when the velocity reaches 0. Based on the preset movement rule, the trajectory planning information, the gantry angle value of the beam angle at the start of the movement, and the gantry angle value of the beam angle at the end of the movement, the gantry movement time may be obtained.

The table movement time refers to a movement time for a table to move from a table angle value at one beam angle to a table angle value at another beam angle. In some embodiments, the table movement time may be determined based on the trajectory planning information and the preset movement rule. More descriptions regarding the preset movement rule may be found in related descriptions hereinabove.

Based on the preset movement rule, the trajectory planning information, the table angle value of the beam angle at the start of the movement, and the table angle value of the beam angle at the end of the movement, the table movement time may be obtained.

In some embodiments, the determining the at least one beam-to-beam movement time based on the sorted beam angle sequence includes determining the beam-to-beam movement time between every two adjacent beam angles in the sorted beam angle sequence as the at least one beam-to-beam movement time.

Exemplarily, the sorted beam angle sequence is set to be (α₂, α₃, α₁, α₄), then the at least one beam-to-beam movement time includes a beam-to-beam movement time for the beam to move from the beam angle α₂ to the beam angle α₃, a beam-to-beam movement time for the beam to move from the beam angle α₃ to the beam angle α₁, and a beam-to-beam movement time for the beam to move from the beam angle α₁ to the beam angle α₄. That is to say, the at least one beam-to-beam movement time includes three beam-to-beam movement times.

In 520, at least one movement cost between beams corresponding to a candidate angle set is determined based on the at least one beam-to-beam movement time.

In some embodiments, the processing device may determine each of the at least one beam-to-beam movement time as each of the at least one beam-to-beam movement cost. For example, each of the three beam-to-beam movement times determined in the above embodiment is determined as the movement cost between beams corresponding to the candidate angle set. That is to say, the candidate angle set corresponds to three movement costs between beams.

Further, the processing device may obtain a penalty term of a field execution time based on at least one movement cost between beams corresponding to the candidate angle set. Exemplarily, the penalty term of the radiation field execution time may be expressed by the following Equation (15):

$${F}_{b e a m} = {\sum }_{( i , j ) ∈ R} {L}_{ij} \qquad ( 15 )$$

Lᵢⱼ denotes the beam-to-beam movement time from beam angle i to beam angle j, and R denotes an adjacent angle set. More descriptions regarding the adjacent angle set may be found in related descriptions hereinabove.

In some embodiments of the present disclosure, the beam-to-beam movement time may be determined based on the trajectory planning information, which allows for determining a planned route for a collision-free trajectory by means of the velocity planning information and the acceleration planning information. Thus, a real execution time situation is reflected and a movement cost between beams that is in line with the actual situation is obtained. The penalty term of the radiation field execution time is constructed based on the movement cost between beams, which helps to efficiently determine an optimal combination of radiation field angles with a short execution time.

FIG. 6 is a schematic diagram illustrating a range and step size determination model according to some embodiments of the present disclosure.

The range and step size determination model is a machine learning model. As shown in FIG. 6, an input of a range and step size determination model 620 includes object image data 611, object delineation data 612, and radiotherapy prescription data 613, and an output of the range and step size determination model 620 includes a preset angle range 631 and a preset step size 632.

In some embodiments, the range and step size determination model may be obtained through training. In some embodiments, as shown in FIG. 6, the range and step size determination model 620 may be trained based on a plurality of first training samples 640 with first labels. For example, the processor 120 or the training module 240 may input the plurality of first training samples 640 with the first labels into an initial range and step size determination model, determine a value of a loss function based on a result of the first labels and the initial range and step size determination model, and update, by an iteration, parameters of an initial range and step size determination model based on the value of the loss function. When the loss faction of the initial range and step size determination model meets a first end condition for ending the training, the model training is completed, and a trained range and step size determination model is obtained. The first training sample 640 may include sample image data, sample delineation data, and sample prescription data. The sample image data, the sample delineation data, and the sample prescription data may be determined based on object image data, object delineation data, and radiotherapy prescription data of a historical object, respectively. For example, the object image data of a plurality of historical objects are designated as the sample image data, the object outline data of the plurality of historical objects are designated as the sample delineation data, and the radiotherapy prescription data of the plurality of historical objects are designated as the sample prescription data. The first labels may be an angle range and a step size corresponding to an angle set used in a historical radiation treatment process of the historical object corresponding to the first training sample 640. The first labels may be determined by manual labeling or automated labeling by the system. The first end condition may be that the loss function converges (e.g., a mean square error of the loss function is less than a first error threshold), a count of the iteration reaches a first threshold, etc.

FIG. 7 is a schematic diagram illustrating an evaluation model according to some embodiments of the present disclosure.

The evaluation model is a machine learning model. As shown in FIG. 7, an input of the evaluation model 720 includes a candidate angle set 711 and a target dose distribution 712, and an output of the evaluation model 720 includes at least one movement cost between beams corresponding to the candidate angle set 711.

In some embodiments, the evaluation model may be obtained by training. In some embodiments, the evaluation model 720 may be obtained by training based on a plurality of second training samples 740 with first labels, as shown in FIG. 7. For example, the processor 120 or the training module 240 may input the plurality of second training samples 740 with second labels into an initial evaluation model, determine a value of a loss function through the second labels and results of the initial evaluation model, and iteratively update parameters of the initial evaluation model based on the value of the loss function. The model training is completed when the loss function of the initial evaluation model satisfies a second end condition for stopping the training, and a trained evaluation model is obtained. The second training sample 740 may include a first sample angle set and a first sample dose distribution. The first sample angle set and the first sample dose distribution may be obtained based on a beam angle set and the target dose distribution used in a historical radiation treatment process. A second label may be the movement cost between beams (e.g., total radiation field movement time for the historical radiation treatment process) for the historical radiation treatment process corresponding to the second training sample 740, and the second label may be determined by manual labeling or automated labeling by the system. The second end condition may be that the loss function converges (e.g., the mean square error of the loss function is less than a second error threshold), a count of iterations of training reaches a second count threshold, etc.

It may be understood that the beam angle set used in the historical radiation treatment process cannot include all subsets of the candidate angle set (all possible beam angle sets). In such a case, in some embodiments, the second training sample 740 may be extended through data augmentation, for example, by adding all possible beam angle sets that have not been used in the historical radiation treatment process to the first sample angle set.

In some embodiments, the input of the evaluation model 720 may also include at least one of object physiological data 713, object pathological data 714, a medical equipment feature 715, or the like, as shown in FIG. 7.

The object physiological data refers to data reflecting physiological features of the object, which may include gender, age, height, weight, etc., of the object. For example, the object physiological data may be “gender: male, age: 41, height: 170 cm, weight: 68 kg”.

The object pathological data refers to data reflecting pathological characteristics of the object. The object pathological data may include a shape of the object's lesion, a location of the lesion, a size of the lesion, a malignancy level, etc. For example, the object pathological data may be “a cross-sectional roundness value of the lesion is 0.6 cm, a location of the lesion is the stomach, a size of the lesion is 100 cm³, and a malignancy level grade of the lesion is grade 3”. The shape of the lesion, the location of the lesion, and the size of the lesion may be determined based on the object image data (e.g., a CT image) and the object delineation data.

The medical equipment feature refers to data reflecting an operational state of a medical device (e.g., the medical device 110). The medical equipment feature may include an operational characteristic of the medical device, remaining usage life, a historical count of failures, etc.

The operational characteristic refers to data reflecting a stability degree of the medical device during operation. The operational characteristic may be characterized by an operational stability level (e.g., an operational stability level may include level 1, level 2, and level 3 . . . , which characterize a decreasing stability degree). The operational characteristic may be determined based on a sound frequency when the medical device is working. For example, the processor 120 may determine a ratio of the sound frequency during operation of the medical device to a sound frequency during normal operation. When the ratio is within a range of [98%, 102%], the operational stability level is at level 1, and when the ratio is within a range of [95%, 98%] or within a range of [102%, 105%], the operational stability level is at level 2, etc. The sound frequency during normal operation may be preset, and the sound frequency during the operation of the medical device may be obtained based on an acoustic sensor.

Exemplarily, the medical equipment feature may be that the device operates at a stability level of 1, has a remaining usage life of 5 years, and has a historical count of failures of 3.

In some embodiments of the present disclosure, determining at least one movement cost between beams corresponding to the candidate angle set by the evaluation model may evaluate the candidate angle set comprehensively and accurately, further making the finally selected optimized field angle set have a good treatment effect on the patient. The input of the evaluation model includes patient data and the medical equipment feature, which may make the model use comprehensive information to improve the accuracy of prediction of the evaluation model.

FIG. 8 is a schematic diagram illustrating a fluence map loss calculation model according to some embodiments of the present disclosure.

The fluence map loss calculation model is a machine learning model. As shown in FIG. 8, an input of the fluence map loss calculation model 820 include a candidate angle set 811 and irradiation parameters 812 under each selectable beam angle in the candidate angle set 811, and an output of the fluence map loss calculation model 820 includes an actual fluence map loss 830 corresponding to the candidate angle set 811.

In some embodiments, the fluence map loss calculation model 820 may be obtained by training a plurality of third training samples 840 with third labels, as shown in FIG. 8. For example, the processor 120 or the training module 240 may input the plurality of third training samples 840 with the third labels into an initial fluence map loss calculation model, determine a value of a loss function through the third labels and results of the initial fluence map loss calculation model, and update, by iteration, parameters of the initial fluence map loss calculation model based on the value of the loss function. The model training is completed when the loss function of the initial fluence map loss calculation model satisfies a third end condition for stopping training, and a trained fluence map loss calculation model is obtained. The third training sample 840 may include a second sample angle set and sample irradiation parameters under each selectable beam angle in the second sample angle set. The second sample angle set may be obtained based on a beam angle set used for a historical radiation treatment process, and the sample irradiation parameters under each selectable beam angle in the second sample angle set may be manually set. A third label may be a historical fluence map loss of the historical radiation treatment process corresponding to the third training sample 840, which may be determined manually or by automatic labeling by the system. The third end condition for stooping training may be that the loss function converges (e.g., the mean square error of the loss function is less than a third error threshold), a count of the iteration reaches a historical count threshold, etc.

In some embodiments, the third training sample 840 may be extended through data augmentation, for example, by adding all possible beam angle sets that have not been used in the historical radiation treatment process to the second sample angle set.

In some embodiments, an input of the fluence map loss calculation model may also include at least one of object physiological data 813 and a medical equipment feature 815, as shown in FIG. 8. More descriptions regarding the object physiological data and the medical equipment feature may be found in related descriptions hereinabove.

In some embodiments of the present disclosure, the accuracy of the fluence map loss corresponding to the candidate angle set may be improved by using the fluence map loss calculation model to determine the fluence map loss. The accuracy of the fluence map loss output by the fluence map loss calculation model may be further improved when the input of the fluence map loss calculation model also includes the object physiological data and the medical device feature.

It should be noted that the foregoing descriptions of the process 400, the process 500, the evaluation model 620, the range and step size determination model 720, and the fluence map loss calculation model 820 are intended to be exemplary and illustrative only and do not limit the scope of application of the present disclosure. For those skilled in the art, the process 400, the process 500, the evaluation model 620, the range and step size determination model 720, and the fluence map loss calculation model 820 may be subjected to a variety of modifications and alternations as guided by the present disclosure. However, these modifications and alternations remain within the scope of the present disclosure.

FIG. 9 is a schematic diagram illustrating a weight determination model in some embodiments of the present disclosure.

The weight determination model is a machine learning model, as shown in FIG. 9. An input of the weight determination model 920 includes a penalty term of a radiation field execution time 911 and a fluence map loss 912. An output of the weight determination model 920 includes a weight of the penalty term of the radiation field execution time 931 and a weight of the fluence map loss 932.

In some embodiments, the weight determination model may be obtained through training. In some embodiments, as shown in FIG. 9, the weight determination model 920 may be trained based on a plurality of fourth training samples 940 with a fourth label. For example, the processor 120 or the training module 240 may input the plurality of fourth training samples 940 with the fourth label into an initial weight determination model, determine a value of a loss function through a result of the fourth label and initial weight determination model, and update, by an iteration, parameters of the initial weight determination model based on the value of the loss function. When the loss function of the initial weight determination model meets a fourth end condition at the end of training, the model training is completed, and a trained weight determination model is obtained. The fourth training sample 940 may include a sample penalty term of a radiation field execution time and a sample fluence map loss. The sample penalty term of the radiation field execution time and the sample fluence map loss may be determined based on a historical penalty term of a radiation field execution time and a historical fluence map loss corresponding to a historical radiation treatment process. The fourth label may be the weight of the penalty term of the radiation field execution time and the weight of the fluence map loss of the fourth training sample 940. The fourth label may be determined by manual labeling or automated labeling by the system. The fourth ending condition may be that the loss function converges (e.g., the mean square error of the loss function is less than a fourth error threshold), a count of the iteration reaches a historical count threshold, etc.

In some embodiments, the fourth training sample 940 may be extended through data augmentation, for example, by adding all possible penalty terms of the radiation field execution time and fluence map losses that have not been used in the historical radiation treatment process to the fourth training sample 940.

FIG. 10 is a flowchart illustrating an exemplary process for determining a target radiation field angle set based on a genetic algorithm according to some embodiments of the present disclosure. In some embodiments, process 1000 may be executed by a processing device (e.g., the processor 120). As shown in FIG. 10, process 1000 includes the following operations.

In 1010, algorithmic parameters of the genetic algorithm are determined.

The genetic algorithm refers to a stochastic global search and optimization manner that simulates biological evolutionary mechanisms. Operations of the genetic algorithm mainly include generation of an initial population, calculation of a fitness of individuals, a selection operation, a crossover operation, a mutation operation, and a judgment of end conditions of an iteration, etc.

The algorithmic parameters of the genetic algorithm may include a population upper limit, a selection probability, a mutation probability, a crossover probability, a current count of iterations, a maximum count of iterations, etc. The algorithmic parameters of the genetic algorithm may be determined based on historical data, expert experience, or other means. Exemplarily, initial values of the algorithmic parameters of the genetic algorithm may be set as follows. The population upper limit is 50, the selection probability is 0.1, the mutation probability is 0.05, the crossover probability is 0.1, the current count of iterations is 0, the maximum count of iterations is 100, etc.

In 1020, a population including individuals not exceeding the population upper limit is generated.

The population refers to a group of a plurality of individuals that simulate things in nature (e.g., a biological population, etc.). In this embodiment, the population corresponds to at least one candidate angle set, and each of the plurality of individuals corresponds to each candidate angle set.

The individual refers to a separate entity within the population (e.g., an organism within the biological population). The individual is used to encapsulate the candidate angle set into the population so that an optimal solution (i.e., the target field angle set) may be selected by outperforming the individual under a constructed population evolutionary rule.

Each individual corresponds to an individual code. The individual code is used to describe the information/characteristic that the individual has.

In some embodiments, the individual encode corresponding to the individual may be a candidate angle set. For example, an individual encode corresponding to individual 1 is a candidate angle set A, an individual encode corresponding to individual 2 is a candidate angle set B, etc.

In some embodiments, a manner for generating the population may include that an equal count of individuals as that of the at least one candidate angle set is generated based on the at least one candidate angle set determined from an alternative angle set. The individual code corresponding to each individual is a candidate angle set in the at least one candidate angle set, and at least one individual constitutes the population. For example, if the at least one candidate angle set includes a candidate angle set A, a candidate angle set B, a candidate angle set C, and a candidate angle set D, four individuals are generated, namely, individual 1, individual 2, individual 3, and individual 4, whose individual codes are any of the candidate angle set A, the candidate angle set B, the candidate angle set C, and the candidate angle set D, respectively (which may or may not be repeated).

In some embodiments, the manner for generating the population may include that a preset count of individuals is generated, and the preset count of individuals constitutes the population. An individual code (e.g., a candidate angle set) corresponding to each individual of the preset count of individuals is initialized accordingly when generating the individual (e.g., generating a candidate angle set by the manner described in operation 320).

The population may also be generated based on any other feasible manners, and the manner provided above is for illustrative purposes only and is not a limitation on its scope.

In 1030, a fitness of the individual in the population is assessed.

The fitness is used to measure a degree of superiority or inferiority of individuals in the population. For example, the smaller the fitness of an individual, the better that individual is. In this embodiment, the fitness of the individual in the population is assessed, i.e., the fitness of each candidate angle set in the at least one candidate angle set is assessed.

In some embodiments, the fitness of the individual may be determined by the following Equation (4) in operation 320 and related descriptions thereof:

$$f = α {F}_{b e a m} + β {F}_{d} \qquad ( 4 )$$

Where ƒ denotes a preset objective function value, i.e., the fitness of the individual. Descriptions regarding the meaning of each parameter in the above equation may be found in operation 320 and related descriptions thereof.

In some embodiments, the fitness of the individual may also be determined by the following Equation (10) in operation 320 and its related description:

$$f = α {F}_{b e a m} + β {F}_{d} + γ {F}_{p} \qquad ( 10 )$$

Where ƒ denotes the preset objective function value, i.e., the fitness of the individual. Descriptions regarding the meaning of each parameter in the above equation may be found in operation 320 and related descriptions thereof.

The fitness of the individual may also be determined based on any other feasible manners, and the manner provided above is for illustrative purposes only and ss not intended to be a limitation on its scope.

In 1040, the population is updated to obtain an updated individual.

Updating the population may include one or more of updating individual codes corresponding to the individuals in the population, eliminating existing individuals in the population, adding new individuals to the population, etc. The purpose of updating the population is to simulate a process in which the individuals in the population undergo a process of survival of the fittest. In this embodiment, updating the population is to update the at least one candidate angle set.

In some embodiments, updating the individual codes corresponding to the individuals in the population may include performing, with a mutation probability, a mutation operation on the individuals in the population. The mutation operation includes updating (by some set rule, e.g., randomly adding or subtracting a fixed angle value) one or more beam angles in the individual codes corresponding to the individual (i.e., the candidate angle set) to one or more new beam angles. The mutation probability refers to the probability of the individuals in the population undergoing the mutation operation. For example, a mutation probability of 30% gives a 30% probability of performing the mutation operation on each individual in the population (i.e., there is a 70% probability for each individual does not need to undergo the mutation operation).

In some embodiments, eliminating existing individuals in the population may include removing individuals with fitness greater than a first fitness threshold from the population. For example, if the first fitness threshold is 3.9, then individuals with fitness greater than 3.9 may be removed from the population (i.e., the candidate angle set corresponding to the removed individuals may be deleted).

In some embodiments, adding new individuals to the population may include selecting with a selection probability a plurality of pairs of individuals; and, for each pair of individuals in the plurality of pairs of individuals, performing a crossover operation with a crossover probability on the pairs of individuals. The crossover operation includes exchanging the one or more beam angles between two individuals. A count of beam angles exchanged between the individuals may be preset, for example, the count may be preset to be one. The selection probability refers to the likelihood that individuals in the population may be selected into any of the plurality of pairs of individuals. For example, if the selection probability is 20%, the probability that each individual in the population may be selected into any of the plurality of pairs of individuals is 20% (i.e., there is an 80% probability that each individual may not be selected into any of the plurality pairs of individuals). The crossover probability refers to the likelihood that each pair of individuals in the plurality of pairs of individuals may undergo the crossover operation. For example, if a crossover probability is 80%, the probability that each pair of individuals of the plurality of pairs of individuals may undergo the crossover operation is 80% (i.e., there is an 80% probability that each pair of individuals may not undergo the crossover operation).

For example, in the case where the individuals (i.e., the candidate angle set) include A, B, C, D, E, F, G, H, I, and J (a total of 10 individuals), with a selection probability of 20%, a crossover probability of 60%, and the count of beam angles exchanged between the individuals is preset to be 1, it is determined, based on the selection probability of 20%, whether each of the above 10 individuals is selected into the plurality of pairs of individuals. Assuming that the selected individuals include B, E, G, and I, and that the plurality of pairs of individuals include individuals B-E (a pair of individuals consisting of individual B and individual E) and individuals G-I (a pair of individuals consisting of individual G and individual I), it is determined whether the above pairs of individuals may undergo the crossover operation based on a 60% crossover probability, respectively. Assuming that individual B includes beam angles α₁, α₂, α₃, α₄ and as, and individual E includes beam angles α₆, α₇, α₈, α₉, and α₁₀, and that individuals B-E are determined to undergo the crossover operation. Since the count of beam angles exchanged between individuals is preset to be 1, individual B and individual E each select a beam angle for exchange. Assuming that individual B selects the beam angle α2 and individual E selects the beam angle α₈, individual B after undergoing the crossover operation includes beam angles α₁, α₈, α₃, α₄, and individual E after undergoing the crossover operation includes beam angles as, and α₆, α₇, α₂, α₉, and α₁₀, and other individuals (including individuals A, C, D, F, G, H, I, and J) include beam angles that are unchanged. The selection of the beam angles from the individuals to be crossed may be random or based on a preset selection rule (e.g., selecting a first beam angle between the individuals).

The population may also be updated based on any other feasible manners, and the manner provided above is for illustrative purposes only and is not a limitation on its scope.

In 1050, the algorithm parameters are updated.

Updating the algorithm parameters refers to updating one or more algorithm parameters after each iteration (e.g., after executing operation 1040). For example, after each iteration, make iterx=iterx+1, where iterx denotes a current count of iteration.

In 1060, whether an iteration end condition of the genetic algorithm is satisfied is determined.

In some embodiments, there may be a plurality of iteration end conditions of the genetic algorithm. For example, the current count of iterations reaches a maximum count of iterations, or there exists an individual whose fitness value satisfies a requirement (e.g., less than the first fitness threshold), etc.

In some embodiments, when the iteration end condition of the genetic algorithm is not satisfied, the determination module 220 may continue to perform the next iteration of iterations (e.g., skip to operation 1030). If the iteration end condition of the genetic algorithm is satisfied, end the iteration and proceed to operation 1070.

In 1070, an individual with an optimal fitness in a current population is determined and output.

The individual with the optimal fitness in the population is the candidate angle set that is optimal in the at least one candidate angle set. In some embodiments, the processing device (e.g., the processor 120) may determine an individual code corresponding to the individual with the optimal fitness in the current population (i.e., the candidate angle set) as the target radiation field angle set and output it.

The target radiation field angle set in the genetic algorithm may also be determined based on any other feasible manners (e.g., based on user requirements), and the manner provided above is for illustrative purposes only and is not a limitation on its scope.

In some embodiments of the present disclosure, when determining the target radiation field angle set based on the genetic algorithm, the population is continuously updated and screened through the fitness of the individuals. Further, an optimized radiation field angle set that meets the user requirements may be quickly determined, thereby improving the efficiency of determining the target radiation field angle set.

FIG. 11 is a flowchart illustrating an exemplary process for determining a target radiation field angle set based on a pruning optimization algorithm according to some embodiments of the present disclosure. In some embodiments, process 1100 may be executed by a processing device (e.g., the processor 120). As shown in FIG. 11, the process 1100 includes the following operations.

In 1110, a root node is generated based on an alternative angle set.

The pruning optimization algorithm refers to a search optimization algorithm that avoids some unnecessary traversal search processes by some kind of judgment. The pruning optimization algorithm searches for an optimal solution by constructing one or more pruning optimization trees by adding nodes one by one. The pruning optimization algorithm mainly include generation of the pruning optimization tree (generation of root nodes and generation of subordinate sub-nodes), constraint judgment, iteration end condition judgment, etc.

The root node is a starting node of the pruning optimization tree.

In some embodiments, generating the root node based on the alternative angle set may include determining at least one candidate angle set; generating the at least one root node, associating each generated root node with the candidate angle set (e.g., representing the candidate angle set as a node property of the root node). In other words, the at least one candidate angle set corresponds one by one with the at least one root node. For example, if five candidate angle sets are obtained in operation 310, there exist five root nodes, and each of the five root nodes is associated with five candidate angle sets.

The root node may also be determined based on any other feasible manner (e.g., based on user requirements), and the manner provided above is for illustrative purposes only and is not a limitation on its scope.

In 1120, a subordinate sub-node is generated based on a pruning constraint.

The subordinate sub-node refers to a post-generated node that have a connection relationship with a node. For example, a root node a has been generated, at this time a root node b is generated and connected to the root node a (which may be connected by directed edges, a direction of the directed edges is a previously generated node pointing to a newly generated node). The node b is the subordinate sub-node of the root node a, and the process is also referred to as the generation of the subordinate sub-node b of the root node a.

In some embodiments, for each branch of each of the plurality of pruning optimization trees (i.e., a branch corresponding to each root node in the at least one root node), the processing device (e.g., the processor 120) may determine whether there is a beam angle that satisfies the pruning constraint among the remaining selectable beam angles for that branch. If there exists no beam angle that satisfies the pruning constraint, no subordinate sub-node is generated for the branch. If there exists a beam angle that satisfies the pruning constraint, at least one beam angle that satisfies the pruning constraint may be independently added into the candidate angle set corresponding to a leaf node of the branch to generate at least one new candidate angle set, and at least one subordinate sub-node of the branch may be generated based on the at least one new candidate angle set.

The branch is a node sequence in the pruning optimization tree that traverses from the root node to a leaf node. The leaf node refers to a node in the pruning optimization tree that does not have a subordinate sub-node. For example, the pruning optimization tree includes the root node a, the subordinate sub-node b and a subordinate sub-node c of the root node a, a subordinate sub-node d and a subordinate sub-node e of a root node b, and a subordinate sub-node f and a subordinate sub-node g of a root node c. Then, the root node a, the subordinate sub-node b of the root node a, the subordinate sub-node d of the root node b (the subordinate sub-node d is the leaf node) constitute a branch (which may be denoted as a→b→d), the root node a, the subordinate sub-node c of the root node a, the subordinate sub-node g of the root node c (where the subordinate sub-node g is a leaf node) and their connection relationship (e.g., edges) form a branch, etc. (which may be denoted as a→c→g).

The remaining selectable beam angles of the branch refer to the remaining selectable beam angles in an alternative angle set, excluding the beam angle in the candidate angle set corresponding to the current leaf node of the branch. For example, there are a total of eight selectable beam angles in the alternative angle set (denoted as α₁, α₂, . . . , α₈). If the leaf node of a branch corresponds to a candidate angle set of A1={α₂, α₅, α₇}, then the remaining selectable beam angles of the branch are α₁, α₃, α₄, α₆, and α₈. When generating the subordinate sub-node from the root node, a count of beam angles corresponding to the subordinate sub-node is always greater than a count of beam angles corresponding to a superior sub-node.

The candidate angle set corresponding the leaf node refers to a candidate angle set that has an association relationship with the leaf node (e.g., the association relationship is represented as a node attribute).

Generating the subordinate sub-node of a branch refers to generating the subordinate sub-node of the leaf node of the branch. The leaf node of a branch refers to a node of the branch that has no subordinate sub-node. For example, for branch a→b→d, node d is a leaf node of the branch. As another example, for branch a, the root node a is a leaf node of the branch, i.e., at this point, there is only one root node a in the current branch.

The pruning constraint is used to determine whether to generate the subordinate sub-node of the branch.

For example, the pruning constraint may be that a candidate angle set corresponding to the leaf node of the current branch does not completely cover the target region and that the coverage of the new candidate angle set on the target region increases after adding a beam angle of the remaining selectable beam angles of the branch to the candidate angle set. Merely by way of example, taking the pruning constraint as an example, let the candidate angle set corresponding to the leaf node of a branch to be A1={α₂, α₅, α₇}, then the remaining selectable beam angles of the branch are α₁, α₃, α₄, α₆, and α₈. If the candidate angle set A1 does not completely cover the target region, and the coverage of the new candidate angle sets {α₂, α₅, α₇, α₃} and {α₂, α₅, α₇, α₆} on the target region increases after adding the beam angles α₃ and α₆ into the candidate angle set A1, in the remaining selectable beam angles of the branch, the beam angles α₃ and α₆ are the beam angles that satisfy the pruning constraint.

As another example, the pruning constraint may be that a preset objective function value of a candidate angle set corresponding to the leaf node of the current branch is greater than a second function value threshold (the smaller the preset objective function value, the better the pruning constraint is), and the preset objective function value of the new candidate angle set decreases or decreases by a degree greater than a decrease degree threshold after adding a beam angle in the remaining selectable beam angles of the branch to the candidate angle set.

The pruning constraint may also be determined based on any other feasible manners (e.g., based on user requirements), and the manner provided above is for illustrative purposes only and is not a limitation on its scope.

The process of generating the subordinate sub-node of a branch based on the candidate angle set may include generating the subordinate sub-node of the branch and associating each of the generated subordinate sub-nodes with a candidate angle set (e.g., the new candidate angle set), such as representing the new candidate angle set as a node property.

For example, the candidate angle set corresponding to the leaf node of a branch is A1={α₂, α₅, α₇}, and the remaining selectable beam angles of the branch are α₁, α₃, α₄, α₆, and α₈. If the beam angles α₃ and α₆ in the remaining selectable beam angles satisfy the pruning constraint, then the beam angles α₃ and α₆ are independently added into the candidate angle set corresponding to the leaf node of the branch to generate two new candidate angle sets {α₂, α₅, α₇, α₃} and {α₂, α₅, α₇, α₆}, respectively. Further, the processing device may generate two subordinate sub-nodes of the branch and associate the two newly generated subordinate sub-nodes, respectively, with the two new candidate angle set as described previously.

In 1130, whether an iteration end condition of the pruning optimization algorithm is satisfied is determined.

In some embodiments, there may be a plurality of iteration end conditions of the pruning optimization algorithm. For example, the iteration end condition of the pruning optimization algorithm may include that all branches of the pruning optimization tree are unable to continue to add the subordinate sub-node, or that there exists a leaf node of the pruning optimization tree corresponding to a candidate angle set with a count of radiation field and a preset objective function value that meet requirements (e.g., the count of radiation field is greater than a threshold, the preset objective function value is less than a threshold, etc.). More descriptions regarding the count of radiation fields may be found in FIG. 3 and the related descriptions thereof.

The iteration end condition of the pruning optimization algorithm may also be determined based on any other feasible manner (e.g., based on user requirements), and the manner provided above is for illustrative purposes only and is not a limitation on its scope.

If the iteration end condition of the pruning optimization algorithm is not satisfied, skip to operation 1120. If the iteration end condition of the pruning optimization algorithm is satisfied, proceed to operation 1140. For example, if the iteration end condition of the pruning optimization algorithm is not satisfied, the processing device may continue to determine, for each branch of each pruning optimization tree, whether there exists, among the remaining selectable beam angles of the branch, a beam angle that satisfies the pruning constraint. If there is such beam angle, at least one beam angle satisfying the pruning constraint is independently added into the candidate angle set corresponding to the leaf node of the branch to generate at least one new candidate angle set and generate at least one subordinate sub-node of the branch based on the at least one new candidate angle set. If there is no such beam angle, the subordinate sub-node of the branch is not generated.

In 1140, the target radiation field angle set is determined.

In some embodiments, at the end of the iteration, the processing device may compare the preset objective function values of the candidate angle set corresponding to the leaf node of each pruning optimization tree and determine the candidate angle set with the largest preset objective function value as the target radiation field angle set.

The target radiation field angle set in the pruning optimization algorithm may also be determined based on any other feasible manners (e.g., based on user requirements), and the manner provided above is for illustrative purposes only and is not a limitation of its scope.

In some embodiments of the present disclosure, when determining the target radiation field angle set based on the pruning optimization algorithm, a search space may be effectively reduced by cutting out some branches that do not satisfy the pruning constraint to improve searching efficiency, which may, in turn, to improve the efficiency of determining the target radiation field angle set.

FIG. 12 is a flowchart illustrating an exemplary process for determining a target radiation field angle set based on a generation optimization algorithm according to some embodiments of the present disclosure. In some embodiments, process 1200 may be executed by a processing device (e.g., the processor 120). As shown in FIG. 12, the process 1200 includes the following operations.

In some embodiments, determining the target radiation field angle set based on the generation optimization algorithm may include the following operations.

In 1210, a radiation field impact factor for each unselected beam angle in an alternative angle set is calculated. In some embodiments, operation 1210 may be performed by the processor 120. The unselected beam angle refers to a beam angle in the alternative angle set that has not been selected to be included in a provisional angle set. The provisional angle set refers to an angle set used to hold one or more beam angles selected from the alternative angle set in a process of determining a preferred radiation field angle set based on the generation optimization algorithm. The provisional angle set may be empty before performing the generation optimization algorithm.

The radiation field impact factor is used to characterize the degree to which the unselected beam angle affects the preset objective function value of the provisional angle set.

In some embodiments, the radiation field impact factor of the beam angle may be determined based on a preset objective function value of a provisional angle set before adding the beam angle and a preset objective function value of a provisional angle set after adding the beam angle. For example, the radiation field impact factor of the beam angle may be a difference between the preset objective function value of the provisional angle set after adding the beam angle and the preset objective function value of the provisional angle set before adding the beam angle. Exemplarily, the radiation field impact factor of the beam angle α₁ may be determined by the following Equation (16):

$$z = {f}^{*} - {f}^{′} \qquad ( 16 )$$

Where v denotes the radiation field impact factor of the beam angle α₁, v denotes the preset objective function value of the provisional angle set after adding the beam angle α₁, and ƒ′ denotes the preset objective function value of the provisional angle set before adding the beam angle α₁.

The radiation field impact factor may also be determined based on any other feasible manners (e.g., based on user requirements), and the manner provided above is for illustrative purposes only and is not a limitation on its scope.

In 1220, at least one unselected beam angle in the alternative angle set is added to the provisional angle set based on the radiation field impact factor. In some embodiments, operation 1220 may be performed by the processor 120.

In some embodiments, the processor 120 may add one or more unselected beam angles with a radiation field impact factor satisfying a preset threshold condition in the alternative angle set to the provisional angle set. For example, taking the example that the radiation field impact factor is determined based on the aforementioned equation and is preferable with a smaller preset objective function value, the preset threshold condition may be that the radiation field impact factor is less than a preset factor threshold (determined based on historical data, expert experience, etc.), and the processor 120 may add the unselected beam angle with a radiation field impact factor that is less than the preset factor threshold in the alternative angle set to the provisional angle set. As another example, taking the example that the radiation field impact factor is determined based on the aforementioned equation and is preferable with a smaller preset objective function value, the processor 120 may add the unselected beam angle with the smallest radiation field impact factor in the alternative angle set to the provisional angle set.

The process of determining a beam angle to be added to the provisional angle set based on the radiation field impact factor may also be realized based on any other feasible manners (e.g., realized based on user requirements), and the manner provided above is for illustrative purposes only and is not a limitation on its scope.

In 1230, whether an iteration end condition of the generation optimization algorithm is satisfied is determined. In some embodiments, operation 1230 may be performed by the processor 120.

In some embodiments, there may be a plurality of iteration end conditions of the generation optimization algorithm. For example, there are no unselected beam angles in the alternative angle set, a count of iterations reaches a set upper limit, or a count of beam angles and/or the preset objective function value of the provisional angle set meets requirements (e.g., the count of beam angles is greater than a count threshold, the preset objective function value is less than a third function value threshold, etc.), etc.

The iteration end condition of the generation optimization algorithm may also be determined based on any other feasible manners (e.g., based on user requirements), and the manner provided above is for illustrative purposes only and is not a limitation on its scope.

In response to unsatisfying the iteration end condition of the generation optimization, skip to operation 1210 and continue to calculate the radiation field impact factor of each unselected beam angle in the alternative angle set. In response to satisfying the iteration end condition of the generation optimization, proceed to operation 1240.

In 1240, the target radiation field angle set is determined.

In some embodiments, the processor 120 may determine a current provisional angle set as the target radiation field angle set.

In some embodiments of the present disclosure, when determining the target radiation field angle set based on the generation optimization algorithm, the search space may be effectively reduced by progressively adding a beam angle with the greatest positive impact on a preset objective function, and a target radiation field angle set with a better preset objective function value may be quickly obtained.

The target radiation field angle set may also be determined based on any other feasible algorithm (e.g., particle swarm algorithms, immunization algorithms, etc.), and the algorithm provided above is for illustrative purposes only and is not intended to be a limitation on its scope.

It should be noted that the foregoing descriptions of the process 1000, the process 1100, and the process 1200 are intended to be exemplary and illustrative only, and do not limit the scope of application of the present disclosure. For those skilled in the art, various alternations and variations may be made to the process 1000, the process 1100, and/or the process 1200 under the guidance of the present disclosure. However, these alternations and variations remain within the scope of the present disclosure.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations, therefore, is not intended to limit the claimed processes and methods to any order except as may be specified in the claims. Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the invention. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A system for determining an irradiation field angle, comprising:
at least one storage medium, the at least one storage medium including a set of instructions; and
at least one processor in communication with the at least one storage medium, wherein when executing the set of instructions, the at least one processor is configured to cause the system to perform operations including:
obtaining an alternative angle set, the alternative angle set including multiple selectable beam angles;
determining a target irradiation field angle set based on the alternative angle set through iterative calculations, wherein each iteration of the iterative calculations includes:
determining a preset objective function value related to the alternative angle set, and
determining the target irradiation field angle set based on the preset objective function value.

2. The system of claim 1, wherein the selectable beam angle includes a gantry angle and/or a table angle.

3. The system of claim 1, wherein the selectable beam angle is generated based on a preset angle range and a preset step size.

4. The system of claim 3, wherein at least one processor is further configured to:
determining the preset angle range and/or the preset step size based on at least one of object image data, object delineation data, or radiotherapy prescription data.

5. The system of claim 4, wherein the at least one processor is further configured to:
determine the preset angle range and/or the preset step size based on at least one of a shape of a lesion, a size of the lesion, a malignancy level of the lesion, an amount of target tissue and/or target organ, or a dange level of the lesion.

6. The system of claim 1, wherein the preset objective function value is determined based at least on a penalty term of an irradiation field execution time and a fluence map loss;
the at least one processor is configured to cause the system to perform operations including:
obtaining at least one candidate angle set based on the alternative angle set, the candidate angle set including one or more selectable beam angles the penalty term of the irradiation field execution time being determined based on at least one movement cost between beams corresponding to a candidate angle set of the at least one candidate angle set, and the fluence map loss being determined based on an actual dose distribution corresponding to the candidate angle set and a target dose distribution.

7. The system of claim 6, wherein the at least one movement cost between beams corresponding to the candidate angle set is determined by:
obtaining a sorted beam angle sequence by sorting multiple selectable beam angles included in the candidate angle set based on a preset sorting rule; and
determining the at least one movement cost between beams corresponding to the candidate angle set based on the sorted beam angle sequence.

8. The system of claim 7, wherein the determining the at least one movement cost between beams corresponding to the candidate angle set based on the sorted beam angle sequence includes:
determining at least one beam angle interval value based on the sorted beam angle sequence; and
determining the at least one movement cost between beams corresponding to the candidate angle set based on the at least one beam angle interval value.

9. The system of claim 7, wherein the determining the at least one movement cost between beams corresponding to the candidate angle set based on the sorted beam angle sequence includes:
determining at least one beam-to-beam movement time based on the sorted beam angle sequence; and
determining the at least one movement cost between beams corresponding to the candidate angle set based on the at least one beam-to-beam movement time.

10. The system of claim 6, wherein the determining the at least one movement cost between beams corresponding to the candidate angle set includes:
determining the at least one movement cost between beams corresponding to the candidate angle set using an evaluation model based on the candidate angle set and the target dose distribution, wherein the evaluation model is a machine learning model, an input of the evaluation model includes the candidate angle set and the target dose distribution, and an output of the evaluation model includes the at least one movement cost between beams corresponding to the candidate angle set.

11. The system of claim 6, wherein the fluence map loss is determined by:
determining the fluence map loss corresponding to the candidate angle set using a fluence map loss calculation model based on the candidate angle set and irradiation parameters under each selectable beam angle in the candidate angle set, the fluence map loss calculation model being a machine learning model; wherein an input of the fluence map loss calculation model includes the candidate angle set and the irradiation parameters under each selectable beam angle in the candidate angle set, and an output of the fluence map loss calculation model includes the fluence map loss corresponding to the candidate angle set.

12. The system of claim 6, wherein the determining the preset objective function value of the candidate angle set includes:
determining the preset objective function value by a weighted sum of the penalty term of the irradiation field execution time and the fluence map loss of the candidate angle set.

13. The system of claim 12, wherein a weight of the fluence map loss is determined based on an excessive dose of a target tissue and a target organ.

14. The system of claim 13, wherein the excessive dose of the target tissue and the target organ is determined by:
calculating an excessive dose at each voxel point of the target tissue and the target organ based on the actual dose distribution corresponding to the candidate angle set and the target dose distribution; and
determining the excessive dose of the target tissue and the target organ by a weighted sum of the excessive dose at each voxel point, wherein different voxel points of the target tissue and target organ correspond to different weights.

15. The system of claim 6, wherein the determining the preset objective function value of the candidate angle set includes:
determining the preset objective function value as a product of the penalty term of the irradiation field execution time and the fluence map loss of the candidate angle set; or
determining the preset objective function value as a power of the penalty term of the irradiation field execution time of the fluence map loss of the candidate angle set.

16. The system of claim 6, wherein the determining the preset objective function value of the candidate angle set includes:
determining the preset objective function value by applying a preset mapping to the penalty term of the irradiation field execution time and the fluence map loss of the candidate angle set.

17. The system of claim 6, wherein the determining the preset objective function value of the candidate angle set includes:
determining the preset objective function value based on the penalty term of the irradiation field execution time, the fluence map loss of the candidate angle set, and an irradiation filed number regular term.

18. The system of claim 1, wherein the each iteration of the iterative calculations further includes:
obtaining a first candidate angle set by transforming an existing candidate angle set of a current iteration and updating an existing candidate angle set based on the first candidate angle set;
determining the preset objective function value of each candidate angle set in the existing candidate angle set; and
determining a next candidate angle set to enter a next iteration based on the preset objective function value from the existing candidate angle set.

19. A system for determining an irradiation field angle, comprising:
at least one storage medium, the at least one storage medium including a set of instructions; and
at least one processor in communication with the at least one storage medium, wherein when executing the set of instructions, the at least one processor is configured to cause the system to perform operations including:
determination module obtaining an alternative angle set, the alternative angle set including multiple selectable beam angles;
obtaining a dose restriction condition and delineation data of a region of interest; and
determining a target irradiation field angle set by optimizing the alternative angle set based on the dose restriction condition and the delineation data of the region of interest.

20. A method for determining an irradiation field angle, comprising:
obtaining an alternative angle set, the alternative angle set including multiple selectable beam angles;
determining a target irradiation field angle set based on the alternative angle set through iterative calculations, wherein each iteration of the iterative calculations includes:
determining a preset objective function value related to the alternative angle set, and
determining the target irradiation field angle set based on the preset objective function value.
